(19) United States
(12) Patent Application Publication
(10) Pub. No.: US 2024/0065261 A1
(43) Pub. Date: Feb. 29, 2024

(54) PENETRATING CRYOPROTECTION AGENTS AND METHODS OF MAKING AND USING SAME

(71) Applicant: The Research Foundation for The State University of New York, Amherst, NY (US)

(72) Inventors: Bing GONG, Amherst, NY (US); Yulong ZHONG, Buffalo, NY (US)

(21) Appl. No.: 18/258,731

(22) PCT Filed: Dec. 21, 2021
(86) PCT No.: PCT/US21/64711

§ 371 Date: Jun. 21, 2023

Related U.S. Application Data
(60) Provisional application No. 63/128,765, filed on Dec. 21, 2020.

(51) Int. Cl. A01N 1/02; A61K 8/37; A61K 8/60; A61K 8/44; A61K 8/49; C07C 69/33; C07H 15/207; C07H 13/04; A61Q 19/00

(52) U.S. Cl. CPC A01N 1/0221; A01N 1/0284; A61K 8/375; A61K 8/602; A61K 8/44; A61K 8/4913; C07C 69/33; C07H 15/207; C07H 13/04; A61K 8/60; A61Q 19/007

(57) ABSTRACT

Provided are compounds that are suitable for cryopreservation of cells. The compounds are non-toxic, water soluble, and free of any organic solvents. The compounds have the following structure: Formula (I), where n is 1, 2, 3, or 4 and each R is independently H or Formula (II), where each R′ is independently chosen from aliphatic groups, aryl groups, and amino acid groups and at least one R is not H and the asterisked carbon has R or S stereochemistry or a racemate thereof. Also provided are compositions and methods of using the compounds to cryopreserve cell populations.

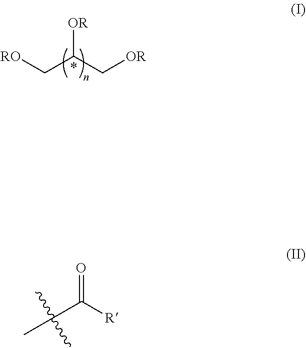

### CROSS REFERENCE TO RELATED APPLICATION

This application claims priority to U.S. Provisional Application No. 63/128,765, filed on Dec. 21, 2020, the disclosure of which is incorporated herein by reference.

### BACKGROUND OF THE DISCLOSURE

Cryopreservation is required in regenerative medicine at nearly all levels and provides the only safe and cost-effective option for the long-term storage of cells, tissues, and organs. Cryoprotective agents (CPAs) are substances used as critical additives that improve the post-thaw viability of cryopreserved biological samples. By preventing ice formation, CPAs help avoid freezing damage to biological samples upon cooling. Two classes of CPAs are known: non-penetrating CPAs that do not enter the cells and act in the extracellular space; or penetrating CPAs that enter the intracellular space. Successful preservation of cells requires the presence of CPAs inside and outside cells. While a variety of substances, including small molecules which cannot penetrate membranes (such as sugars) and large, long chain polymers, have been employed as non-penetrating CPAs, the availability of penetrating CPAs remains very limited, with dimethyl sulfoxide (DMSO), and to a lesser extent glycerol, being the most commonly used penetrating CAP. Unfortunately, DMSO has toxicity, which prevents it from being used for many applications. For preservations that do involve DMSO, cells can only be incubated in DMSO containing cryopreservation solutions in limited to short pre-freeze and post-thaw times. Additionally, DMSO and glycerol are applied in high (10-50% or more) extracellular concentrations in cryopreservation, and require extensive washing during thawing to prevent cell death or subsequent adverse reactions when used in medical treatments. Except for a few membrane-permeable CPAs such as DMSO and glycerol, the majority of known molecules with superb antifreeze properties are impermeable to mammalian cell membranes.

Although many other CPAs have been explored as alternative penetrating CPAs, very few have shown the same efficacy as glycerol or DMSO. Currently there is an urgent need for DMSO-free freezing media since cells are more stable over time in DMSO-free solutions than in DMSO-containing media. As a result, the search for new, effective, non-toxic CPAs that can penetrate cell membranes continues.

Sugar alcohols (polyols) such as glycerol, sorbitol, mannitol, ribitol, erythritol, and threitol, along with several sugars and amino acids, are found to accumulate in most freeze-tolerant insects that survive extremely low temperature in winter. High polyol concentrations (>2 M) have been reported in the body fluids of many species. However, like disaccharides, common sugar alcohols, except for glycerol, are very poor at or incapable of penetrating cell membranes. When being added to cell culture media, these molecules act extracellularly to offer insufficient or no cryoprotection for cells.

### SUMMARY OF THE PRESENT DISCLOSURE

The present disclosure provides esterified polyols. Also provides are methods of making compositions comprising esterified polyols. Also provided are methods of using the esterified polyols and compositions.

The compounds disclosed herein enable the cryopreservation of cells with CPAs that are non-toxic, water soluble, and free of any organic solvents. Substances known to be antifreeze but unable to penetrate cell membranes are modified into membrane-permeable derivatives that are then converted back into their original active forms inside cells. The net outcome is the intracellular delivery of a host of readily available, nontoxic, but are underutilized substances into practically useable, potent intracellular CPAs, realizing DMSO-free cell cryopreservation.

In an aspect, the present disclosure provides esterified polyols. One or more or all of the alcohol groups of the precursor polyol are esterified to form the esterified polyol. The esterified polyols may be referred to as cryoprotective agents (CPAs). The esterified polyols may be referred to as “compounds” throughout.

In an aspect, the present disclosure provides compositions comprising one or more esterified polyols of the present disclosure. The compositions further comprise one or more pharmaceutically acceptable carriers.

In an aspect, the present disclosure provides methods of using one or more esterified polyols of the present disclosure. The one or more esterified polyols may be used in method for preparing a cell population for cryopreservation or in a method of cryopreserving a cell population.

### BRIEF DESCRIPTION OF THE FIGURES

For a fuller understanding of the nature and objects of the disclosure, reference should be made to the following detailed description taken in conjunction with the accompanying figures.

FIG. 1 shows water-soluble, membrane-impermeable A, which carries multiple hydroxyl (OH) groups, is coupled with B, which bears a hydrophobic segment and a carboxyl (COOH) group, to give ester A-B capable of penetrating cell membrane and entering cell. Catalyzed by intracellular esterases, the internalized ester AB is hydrolyzed back into A and B. (A=a water-soluble compound with known antifreeze capability; B=a compound with a hydrophobic segment and with or without antifreeze capability).

FIG. 2 shows coupling sugar alcohols (a) with carboxylic acids (b) leads to esters ESA-1, ESA-2, and ESA-3.

FIG. 3 shows esters ESA-1, ESA-2, and ESA-3 derived from glucose, mannose, and fructose.

FIG. 4 shows esters ESU and ETR derived from sucrose and trehalose.

FIG. 5 shows esters derived from glycerol and sorbitol examined for cryopreservative efficacies.

FIG. 6 shows cell viability data of NIH-3T3 cells cryopreserved with or without GLC3P. Cell viability is obtained with CCK-8 assay, with that from 10% DMSO being set as 100%.

FIG. 7 shows cell viability data of NIH-3T3 cells cryopreserved with or without SBT6P. Cell viability is obtained with CCK-8 assay, with that from 10% DMSO being set as 100%.

FIG. 8 shows cell viability data of NIH-3T3 cells cryopreserved with or without SBT2A-1 and SBT2A-2. Cell viability is obtained with CCK-8 assay, with that from 10% DMSO being set as 100%.

FIG. 9 shows a scheme of acylation of sorbitol and sucrose.

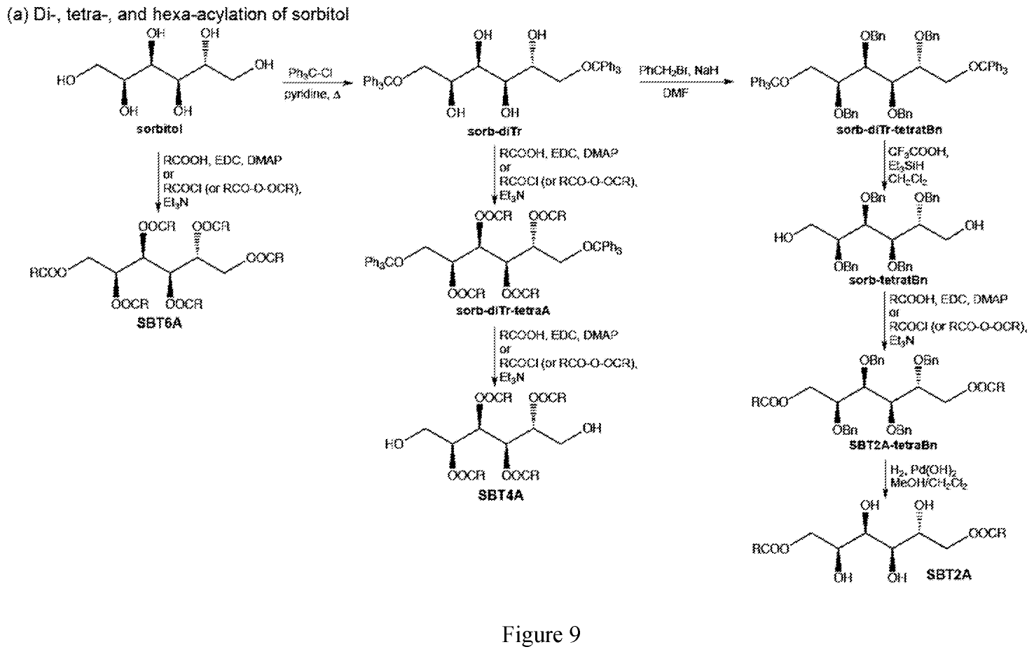

FIG. 10 shows a synthetic scheme of acylation.

FIG. 11 shows average diameters of NIH-3T3 cells incubated with medium only (control), and in media with SorbPr₂ (10 mM) and MannPr₂ (10 mM) (medium=DMEM with 10% FBS) for 48 h. Diameters are expressed as mean±SD based on triplicate measurements. At least 1000 cells per group were analyzed. Statistical analysis was performed with one-way ANOVA, **=p<0.01, ****=p<0.0001.

FIG. 12 shows total cell recovery values of cryopreserved NIH-3T3 cells 0 h and 48 h post-thaw (expressed as mean±SEM based on three independent measurements). The cells were incubated in media with 5 mM and 10 mM of diester SorbPr₂ (10 mM), and also with only medium, followed by freezing in media containing trehalose (400 mM). (media=DMEM+10% FBS).

FIG. 13 shows total cell recovery values of cryopreserved NIH-3T3 cells immediately (0 h), 24 h, and 48 h post-thaw (expressed as mean±SEM based on three independent measurements). The cells were incubated in media with diester MannPr₂, SorbPr₂, XylPr₂, or EryPr₂ (10 mM), and unmodified mannitol, sorbitol, xylitol, or erythritol (10 mM), followed by frozen in media containing trehalose (400 mM). Control-1: Cells incubated with media only then frozen in media containing trehalose (400 mM); Control-2: Cells incubated with media only then frozen in media containing 5% DMSO. (media=DMEM+10% FBS).

FIG. 14 shows fluorescence microscopy images of NIH-3T3 cells 24 h post-thaw, cryopreserved by incubating with (a) mannitol (10 mM), (b) sorbitol (10 mM), (c) media only, (d) MannPr₂ (10 mM), (e) SorbPr₂ (10 mM) for 48 h, and (f) media only, followed by freezing in media (a) to (e) containing 400 mM trehalose and (f) containing 5% DMSO (media=DMEM, 10% FBS). Cells were stained with calcein-AM (green fluorescence, live cells) and ethidium homodimer-1 (red fluorescence, dead cells). Scale bar 100 μm.

FIG. 15 shows cell viability after incubating with a) SorbPr₂ (1), b) SorbBa₂ (1a), c) MannPr₂ (2), d) MannBa₂ (2a), e) XylPr₂ (3), f) EryPr₂ (4) for 48 hours.

FIG. 16 shows cell viability after incubating with a) propionic acid, b) butyric acid for 48 hours.

FIG. 17 shows growth curves of NIH3T3 incubated with medium, SorbPr₂ (10 mM) or MannPr₂ (10 mM). The time course starts at the time of incubating with samples or medium only. The results are the means±SEM based on three independent experiments.

FIG. 18 shows DIC and fluorescence microscopy images of NIH-3T3 cells 24 h post-thaw at lower density cryopreserved by incubating with (b, g) SorbPr₂ (10 mM), (c, h) MannPr₂ (10 mM), (d, i) media only for 48 h, comparing to (a, f) fresh cell and (e, j) cells cryopreserved by 5% DMSO. Cells were stained with calcein-AM (green fluorescence, live cells) and ethidium homodimer-1 (red fluorescence, dead cells). Scale bar 100 μm.

FIG. 19 shows (a) bright field, (b) counted image (green circles) and (c) histogram for one count of cell counting.

FIG. 20 shows cell size histogram of NIH-3T3 cells incubated with (a) medium only (control), (b) SorbPr₂ (10 mM) and (c) MannPr₂ (10 mM).

FIG. 21 shows total cell recovery values of cryopreserved NIH-3T3 cells 48 h post-thaw (expressed as mean±SEM based on three independent measurements). Statistical analysis by one-way ANOVA, **=p<0.01, ***=p<0.001, ****=p<0.0001, ns=no significance.

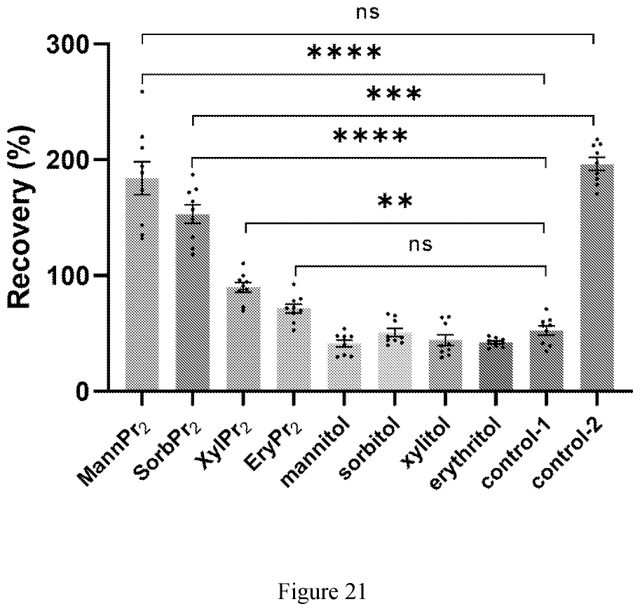

FIG. 22 shows an ¹H NMR spectrum of 1 in DMSO-d₆ at 25° C. (400 MHz).

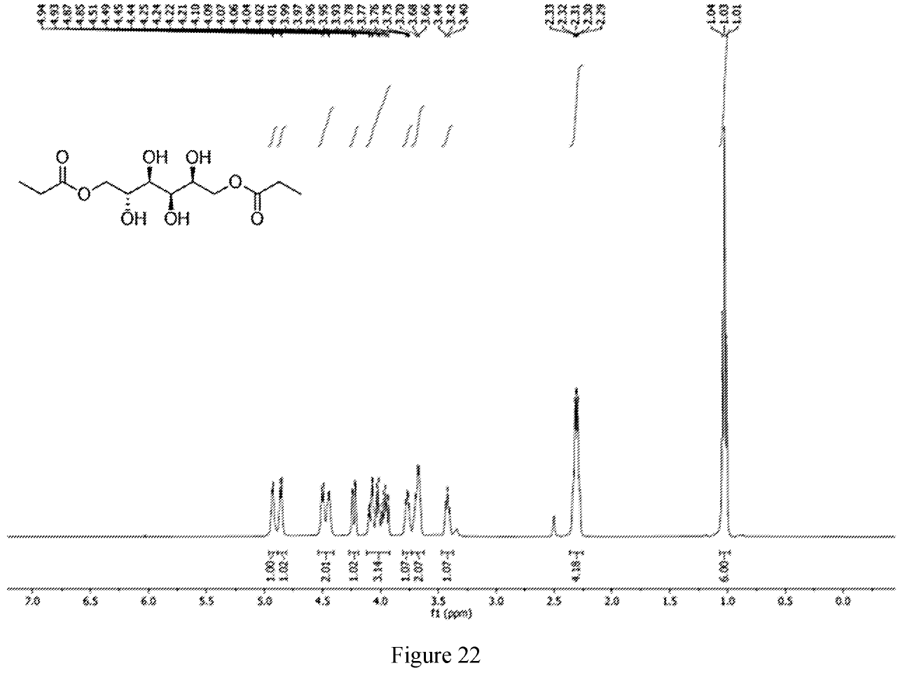

FIG. 23 shows a ¹³C NMR spectrum of 1 in DMSO-d₆ at 25° C. (101 MHz).

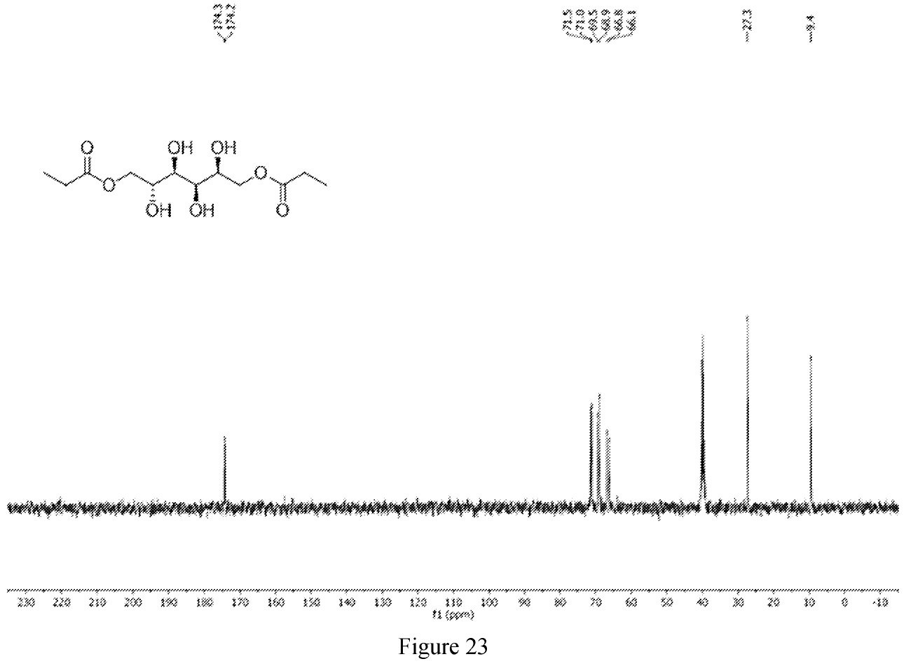

FIG. 24 shows an HRMS spectrum of 1.

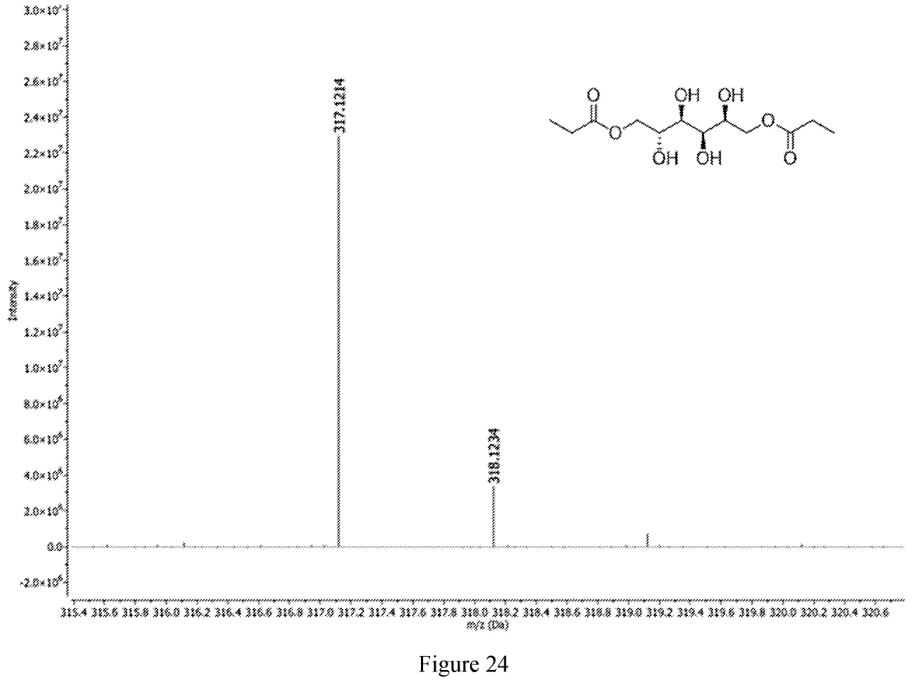

FIG. 25 shows an ¹H NMR spectrum of 1a in DMSO-d₆ at 25° C. (400 MHz).

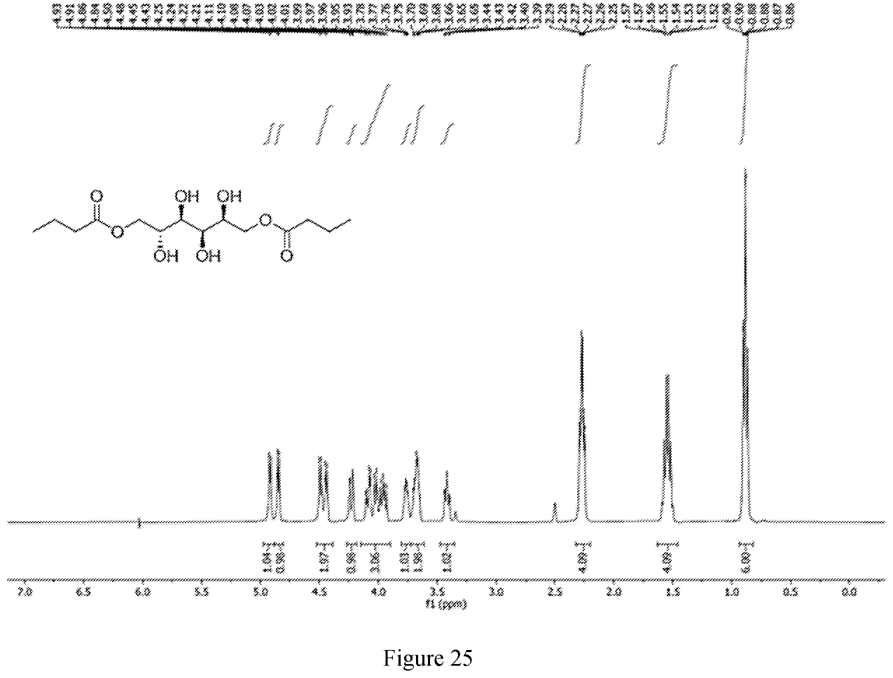

FIG. 26 shows a ¹³C NMR spectrum of 1a in DMSO-d₆ at 25° C. (101 MHz).

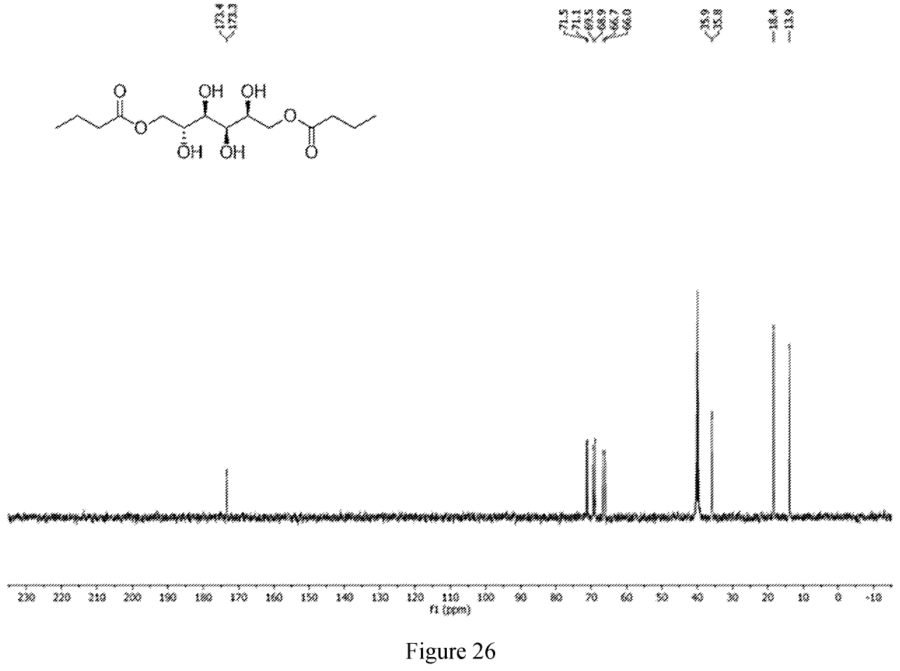

FIG. 27 shows an HRMS spectrum of 1a.

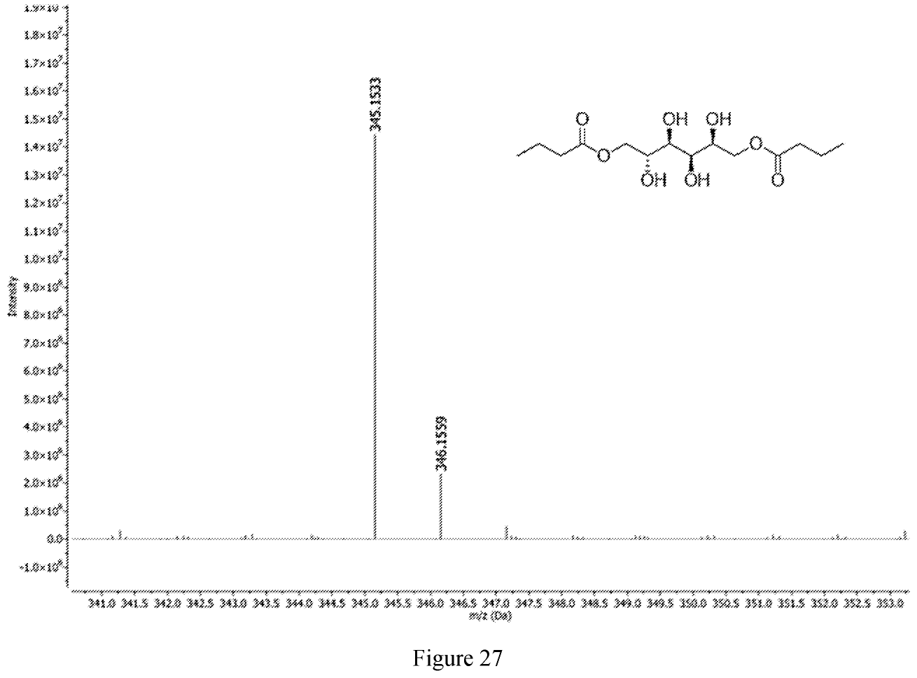

FIG. 28 shows an ¹H NMR spectrum of 2 in DMSO-d₆ at 25° C. (400 MHz).

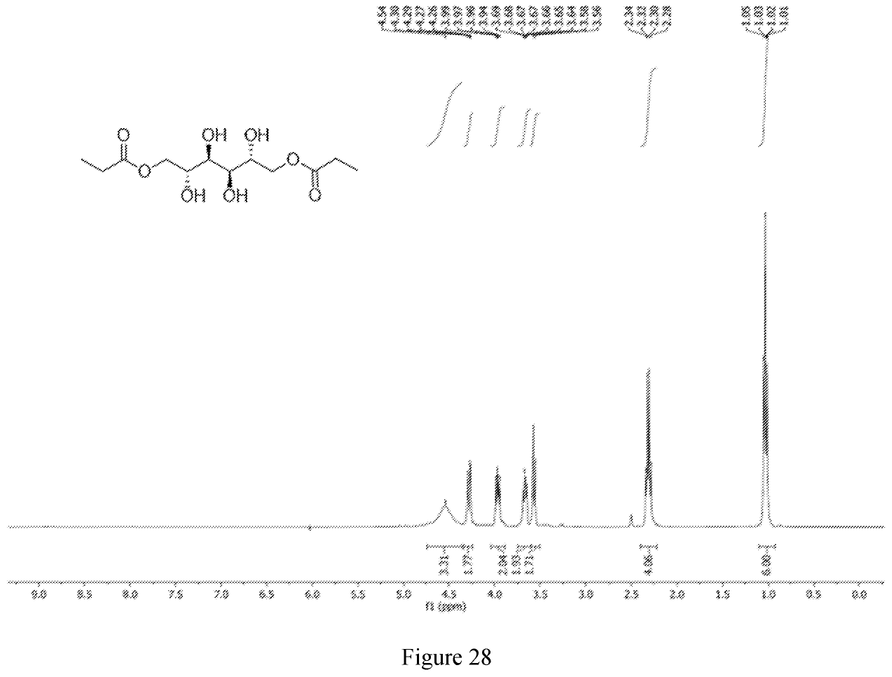

FIG. 29 shows a ¹³C NMR spectrum of 2 in DMSO-d₆ at 25° C. (101 MHz).

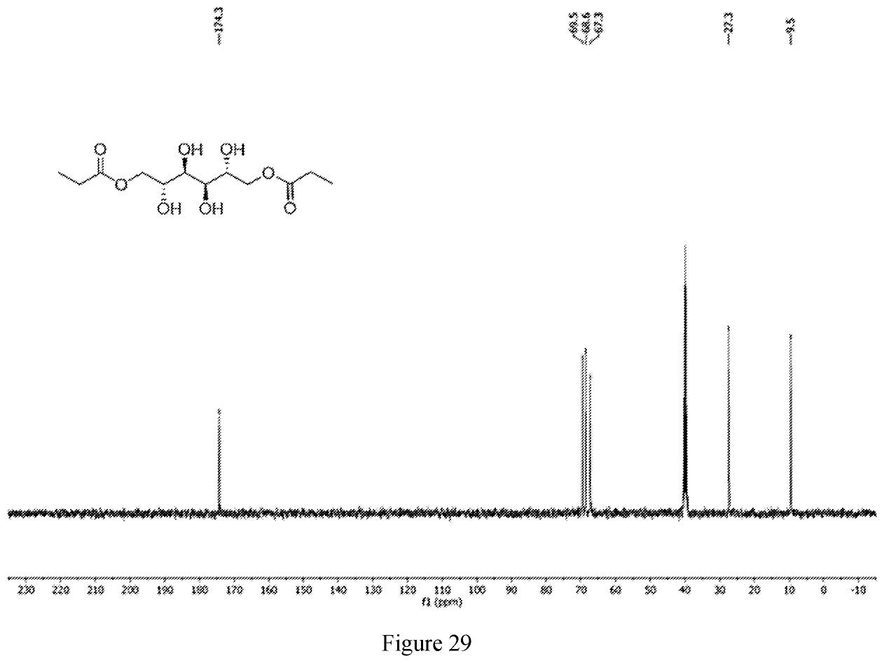

FIG. 30 shows an HRMS spectrum of 2.

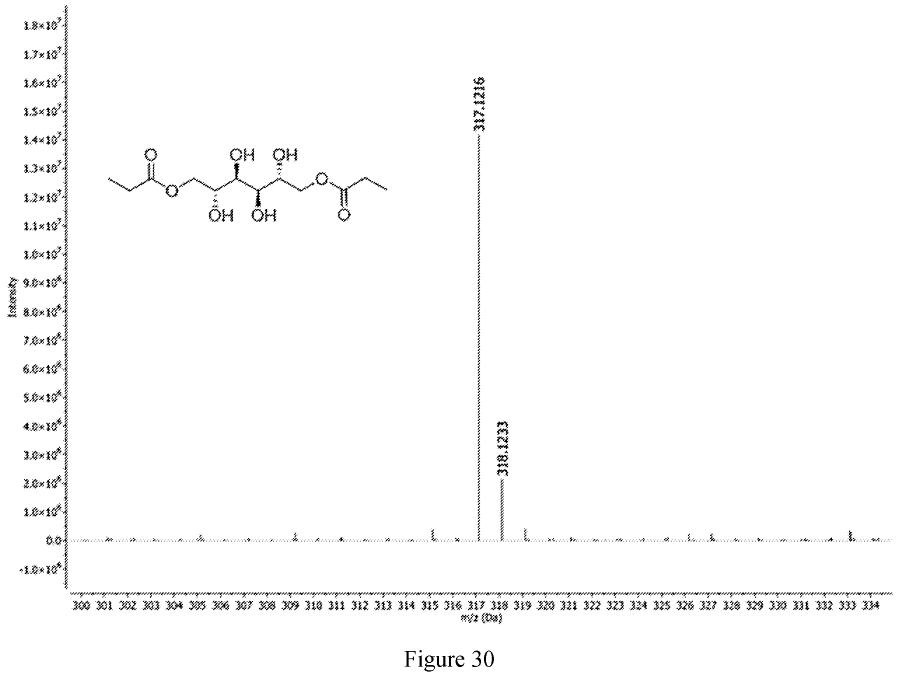

FIG. 31 shows an ¹H NMR spectrum of 2a in DMSO-d₆ at 25° C. (400 MHz).

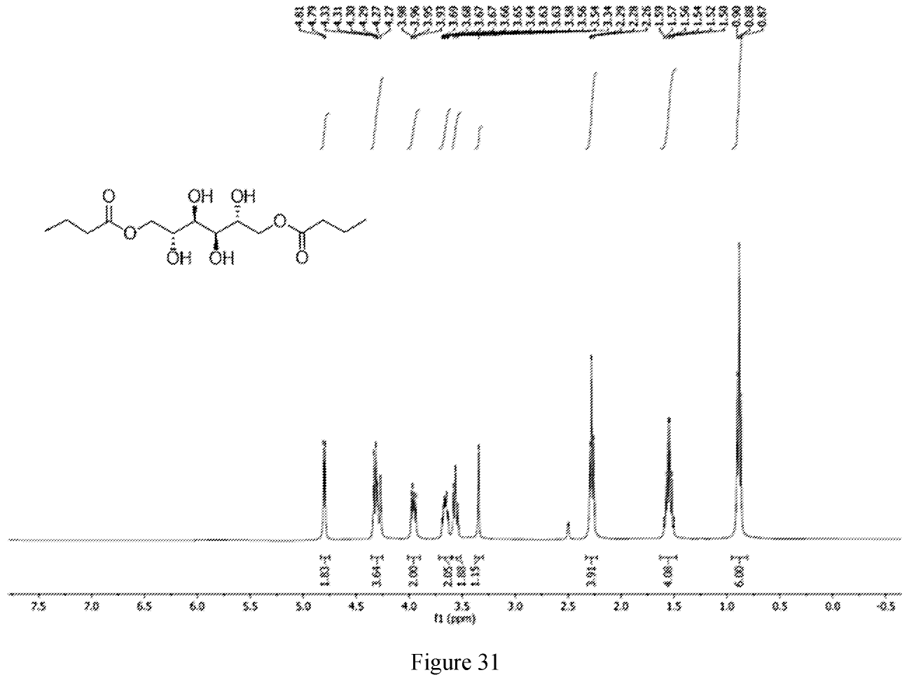

FIG. 32 shows a ¹³C NMR spectrum of 2a in DMSO-d₆ at 25° C. (101 MHz).

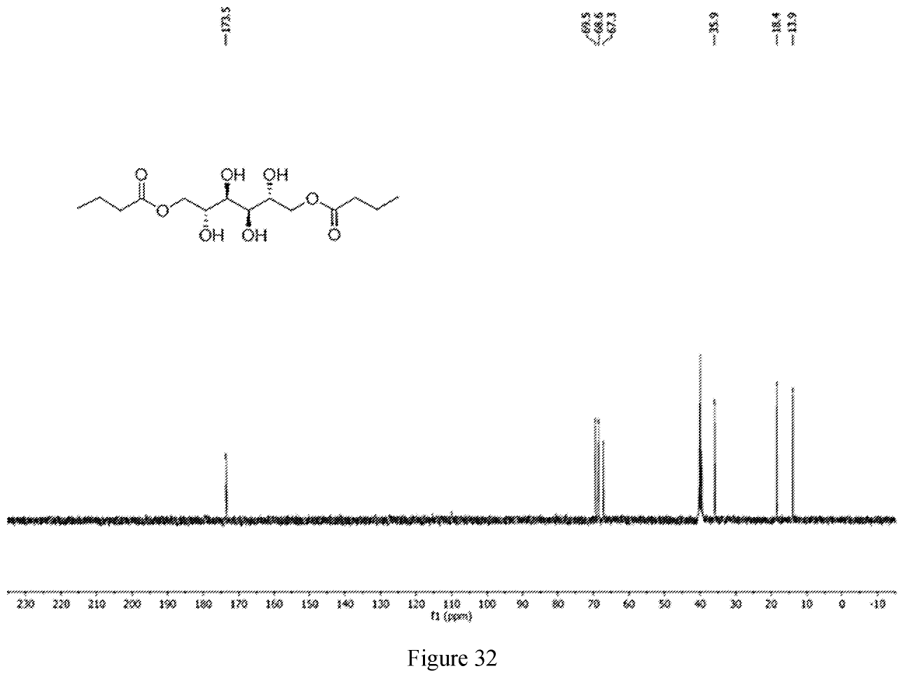

FIG. 33 shows an HRMS spectrum of 2a.

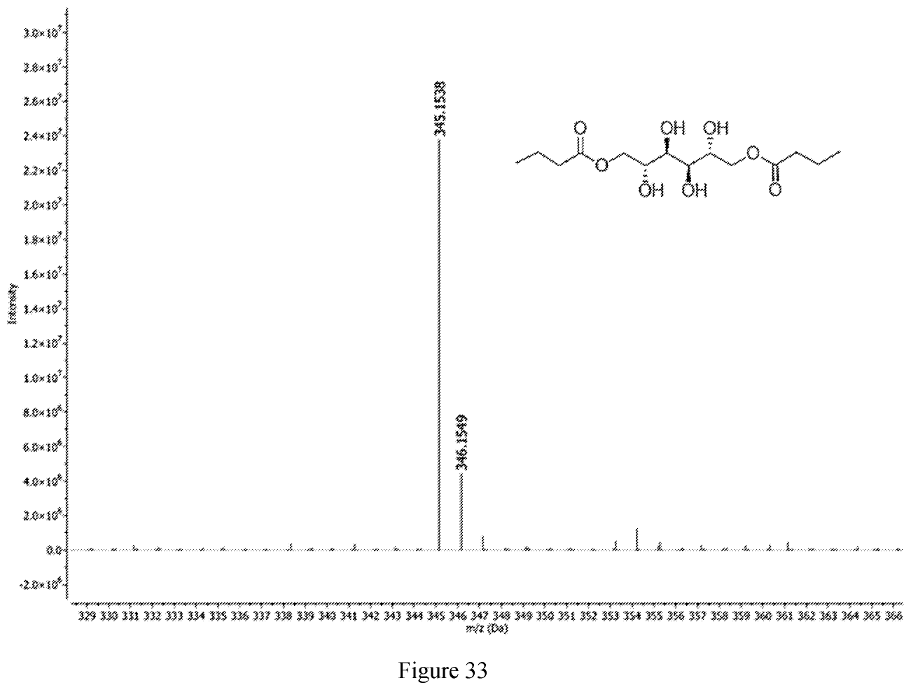

FIG. 34 shows an ¹H NMR spectrum of 3 in DMSO-d₆ at 25° C. (400 MHz).

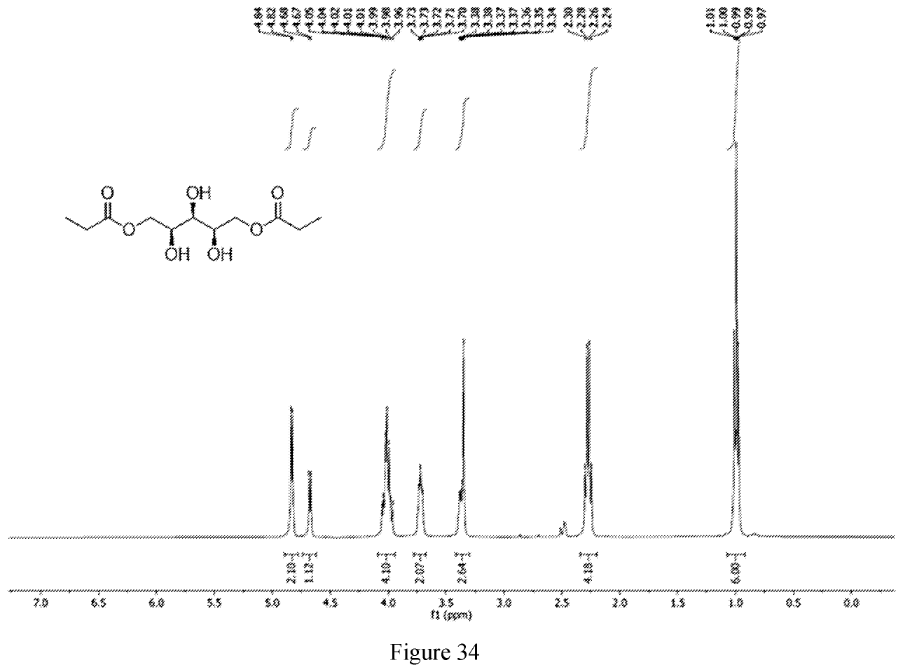

FIG. 35 shows a ¹³C NMR spectrum of 3 in DMSO-d₆ at 25° C. (101 MHz).

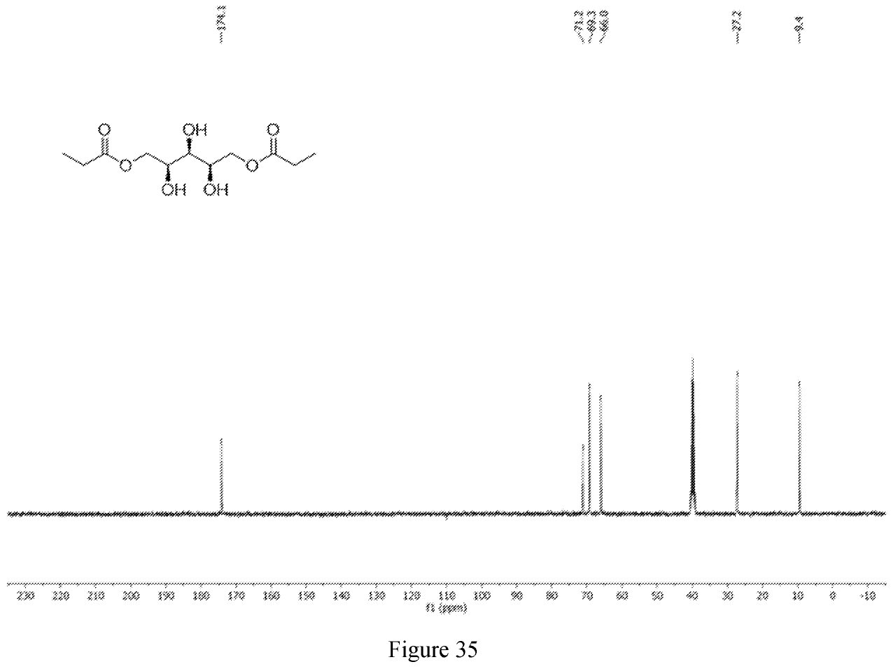

FIG. 36 shows an HRMS spectrum of 3.

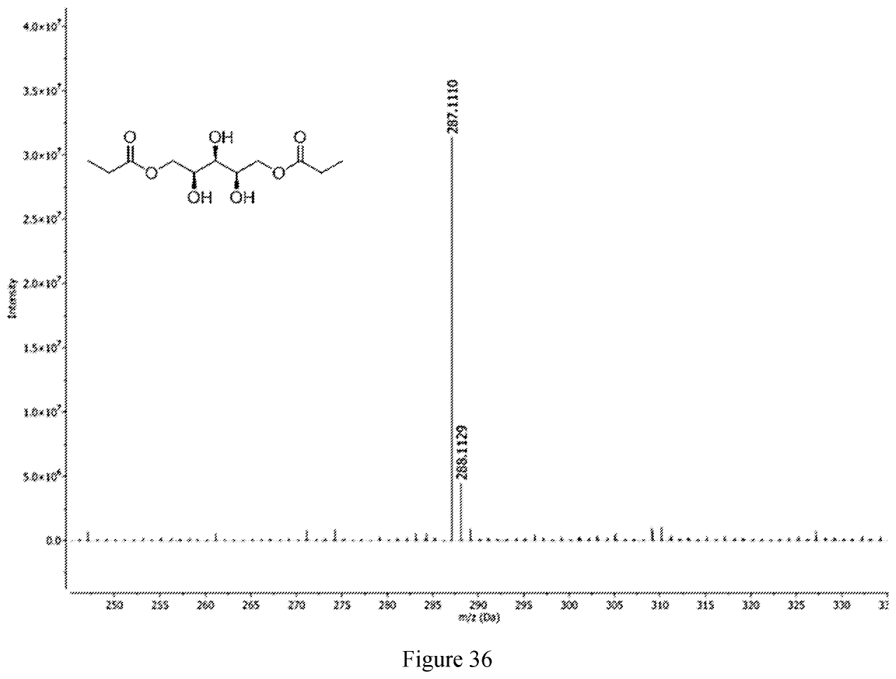

FIG. 37 shows an ¹H NMR spectrum of 4 in DMSO-d₆ at 25° C. (400 MHz).

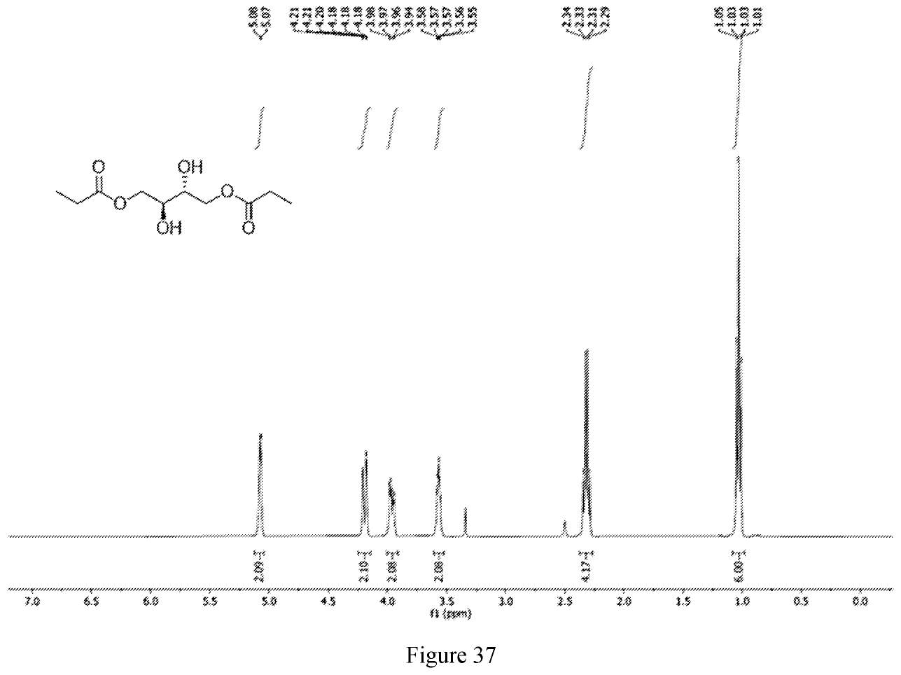

FIG. 38 shows a ¹³C NMR spectrum of 4 in DMSO-d₆ at 25° C. (101 MHz).

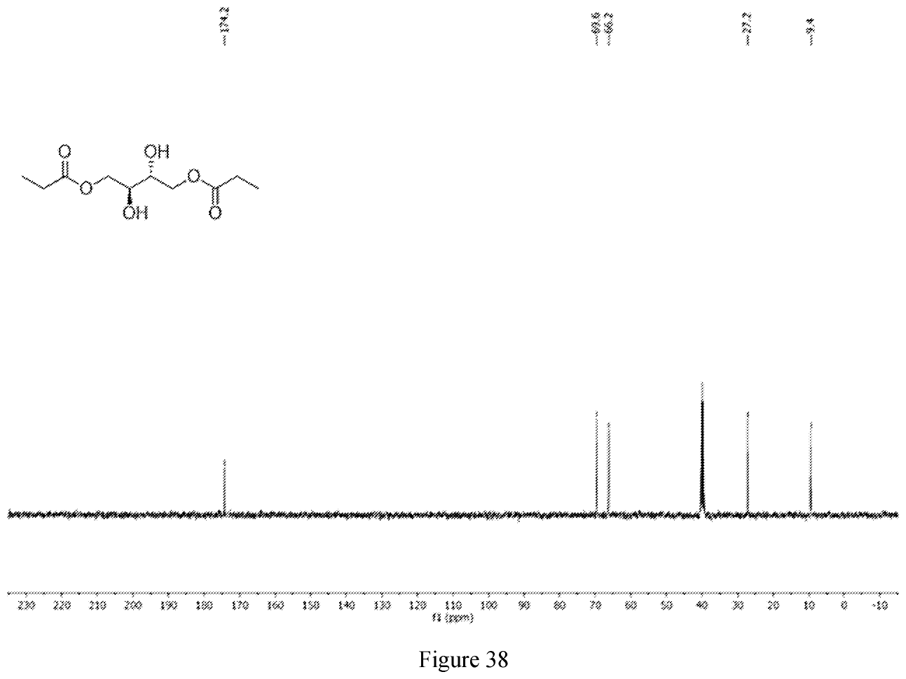

FIG. 39 shows an HRMS spectrum of 4.

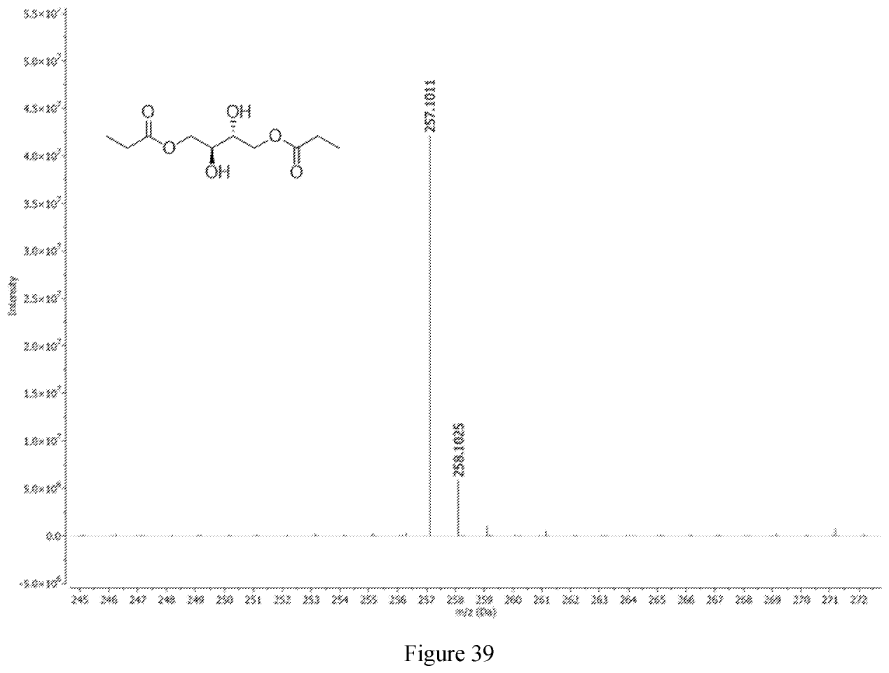

FIG. 40 shows a cartoon illustrating a compound of the present disclosure entering a cell, being subjected hydrolysis by esterase to form a sorbitol or mannitol.

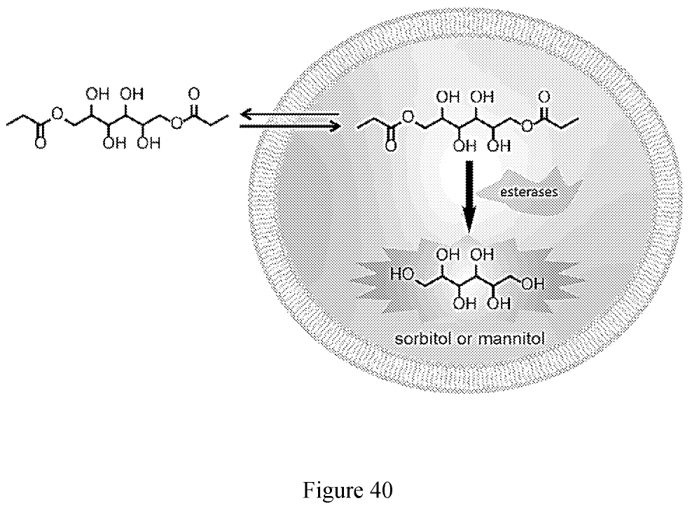

FIG. 41 shows cytotoxicity data of compounds of the present disclosure.

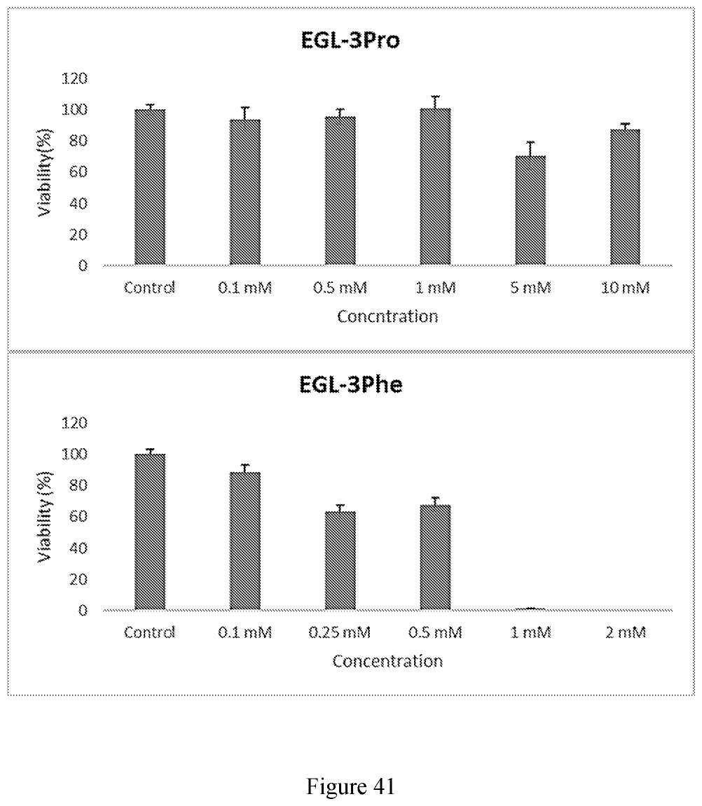

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step changes may be made without departing from the scope of the disclosure.

Ranges of values are disclosed herein. The ranges set out a lower limit value and an upper limit value. Unless otherwise stated, the ranges include all values to the magnitude of the smallest value (either lower limit value or upper limit value) and ranges between the values of the stated range.

As used herein, unless otherwise stated, the term “group” refers to a chemical entity that has one terminus or two or more termini that are covalently bonded to one or more other chemical spec(ies). The term “group” includes radicals (e.g., monovalent and multivalent, such as, for example, divalent, trivalent, and the like, radicals). Examples of groups include, but are not limited to:

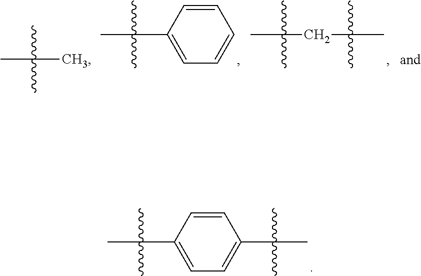

As used herein, unless otherwise indicated, the term “aliphatic groups” refers to branched or unbranched hydrocarbon groups that, optionally, contain one or more degrees of unsaturation. Degrees of unsaturation include, but are not limited to, alkenyl groups, alkynyl groups, and aliphatic cyclic groups. Aliphatic groups may be a C₁ to C₂₀ aliphatic group, including all integer numbers of carbons and ranges of numbers of carbons therebetween (e.g., C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, and C₂₀). Aliphatic groups may be unsubstituted or substituted with one or more substituents. Examples of substituents include, but are not limited to, halogens (—F, —Cl, —Br, and —I), aliphatic groups (e.g., alkyl groups, alkenyl groups, alkynyl groups, and the like), halogenated aliphatic groups (e.g., trifluoromethyl group and the like), aryl groups, halogenated aryl groups, alkoxide groups, amine groups, nitro groups, carboxylate groups, carboxylic acids, ether groups, alcohol groups, alkyne groups (e.g., acetylenyl groups and the like), and the like, and combinations thereof. Aliphatic groups may be alkyl groups, alkenyl groups, alkynyl groups, or carbocyclic groups, and the like.

As used herein, unless otherwise indicated, the term “alkyl groups” refers to branched or unbranched saturated hydrocarbon groups. Examples of alkyl groups include, but are not limited to, methyl groups, ethyl groups, propyl groups, butyl groups, isopropyl groups, tert-butyl groups, and the like. For example, an alkyl group is a C₁ to C₁₂ alkyl group, including all integer numbers of carbons and ranges of numbers of carbons therebetween (e.g., C₁, C₂, C₃, C₄, C₅, C₆, C₇, C₈, C₉, C₁₀, C₁₁, or C₁₂). The alkyl group may be unsubstituted or substituted with one or more substituent(s). Examples of substituents include, but are not limited to, substituents such as, for example, halogens (—F, —Cl, —Br, and —I), aliphatic groups (e.g., alkyl groups, alkenyl groups, alkynyl groups), aryl groups, alkoxide groups, amine groups, carboxylate groups, carboxylic acids, ether groups, alcohol groups, alkyne groups (e.g., acetylenyl groups), and the like, and combinations thereof.

As used herein, unless otherwise indicated, the term “aryl groups” refers to C₅ to C₁₄ (e.g., C₅, C₆, C₇, C₈, C₉, C₁₀, C₁₁, C₁₂, C₁₃, or C₁₄), including all integer numbers of carbons and ranges of numbers of carbons therebetween, aromatic or partially aromatic carbocyclic groups. The aryl groups may comprise (or be) polyaryl groups such as, for example, fused ring or biaryl groups. The aryl group may be unsubstituted or substituted with one or more substituent(s). Examples of substituents include, but are not limited to, substituents such as, for example, halogens (—F, —Cl, —Br, and —I), aliphatic groups (e.g., alkenes, alkynes), aryl groups, alkoxides, carboxylates, carboxylic acids, ether groups, sulfonic acids/sulfonates (which may be present as a salt such as, for example, a Group I cation, Group II cation, ammonium salt, or the like, or a combination thereof) groups, and the like, and combinations thereof. Examples of aryl groups include, but are not limited to, phenyl groups, biaryl groups (e.g., biphenyl groups), and fused ring groups (e.g., naphthyl groups).

The present disclosure provides esterified polyols. Also provides are methods of making compositions comprising esterified polyols. Also provided are methods of using the esterified polyols and compositions.

The compounds disclosed herein enable the cryopreservation of cells with CPAs that are non-toxic, water soluble, and free of any organic solvents. Substances known to be antifreeze but unable to penetrate cell membranes are modified into membrane-permeable derivatives that are then converted back into their original active forms inside cells. The net outcome is the intracellular delivery of a host of readily available, nontoxic, but are underutilized substances into practically useable, potent intracellular CPAs, realizing DMSO-free cell cryopreservation.

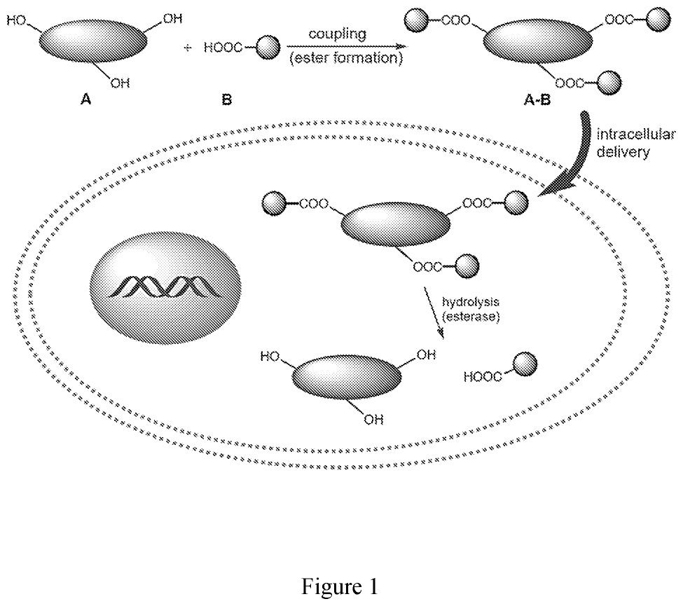

As illustrated in FIG. 1, water-soluble compound A carrying multiple hydroxyl (OH) groups may be coupled to acid B, i.e., a compound bearing a carboxyl (COOH) group, forming ester A-B (which may be referred to as an esterified polyol). Compound A has anti-freeze capability but cannot penetrate the cell membrane. Acid B carries a hydrophobic or a cationic segment and may or may not have anti-freeze capability. Ester A-B has at least modest (>0.1 mM) or good (≥5 mM) solubility in water but is able to cross the cell membrane. Upon entering the intracellular space, ester A-B is hydrolyzed, which is catalyzed by esterases that do not exist in the extracellular culture media, back to A and B. The internalized A, along with compound B if B also has antifreeze ability, acts as an intracellular CPA that prevents ice formation upon freezing and thus leads to the protection of cells.

In an aspect, the present disclosure provides esterified polyols. One or more or all of the alcohol groups of the precursor polyol are esterified to form the esterified polyol. The esterified polyols may be referred to as cryoprotective agents (CPAs). The esterified polyols may be referred to as “compounds” throughout.

A polyol from which the esterified polyol is formed from has two or more alcohol groups. Various polyols may be used to form an esterified polyol. Non-limiting examples of polyols include polyhydric alcohols (which may be referred to as “sugar alcohols”), monosaccharides, and disaccharides.

Various polyhydric alcohols may be used to form an esterified polyol of the present disclosure. A polyhydric alcohol may have the following structure:

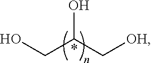

where n is 1, 2, 3, or 4 and the asterisked carbon has R or S stereochemistry or a racemate thereof. Examples of polyhydric alcohols include, but are not limited to glycerol, erythritol, xylitol, mannitol, sorbitol, galactitol, and the like.

An esterified polyol formed from a polyhydric alcohol may have the following structure:

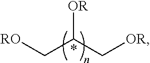

where n is 1, 2, 3, or 4 and each R is independently H or

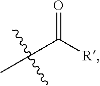

where each R′ is independently chosen from aliphatic groups, aryl groups, and amino acid groups (e.g.,

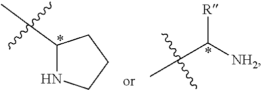

wherein the asterisked carbon has R stereochemistry or S stereochemistry and R″ is the sidechain of a canonical amino acid or the sidechain of a non-canonical amino acid) and at least one R is not H and the asterisked carbon has R or S stereochemistry or a racemate thereof.

Various aliphatic groups may be used. The aliphatic groups may be substituted or unsubstituted and/or linear or branched aliphatic groups. Non-limiting examples of aliphatic groups include a CH₃ group, a C₂H₅ group, linear and branched C₃H₇ groups, linear and branched C₄H₉, C₅H₁₁, and C₆H₁₃ groups, and the like. In various examples, the aliphatic group is a C₃H₇ group.

Various aryl groups may be used. The aryl groups may be substituted or unsubstituted aryl groups. Non-limiting examples of aryl groups include phenyl groups; and

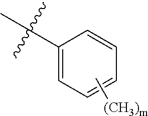

groups, where m is 1, 2, 3, 4, or 5; and the like.

Amino acid groups are formed from amino acids (e.g.,

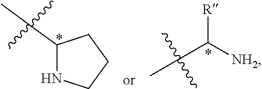

where the asterisked carbon has R stereochemistry or S stereochemistry and R″ is the sidechain of a canonical amino acid or the sidechain of a non-canonical amino acid). The amino acids may be canonical amino acids or non-canonical amino acids. The amino acids may be derivatives of glycine (e.g., a glycine functionalized at the amine). Examples of

amino acid groups include, but are not limited to, prolinyl groups

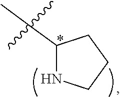

glycine betainyl groups

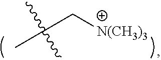

glycocyaminyl groups

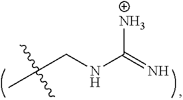

and the like. An esterified polyol formed from a polyhydric alcohol may have the following structure:

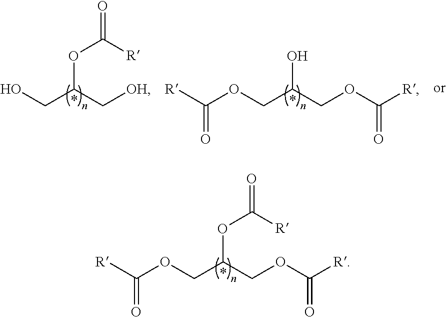

where the asterisked carbon has R or S stereochemistry or a racemate thereof. In various examples, the esterified polyol has the following structure:

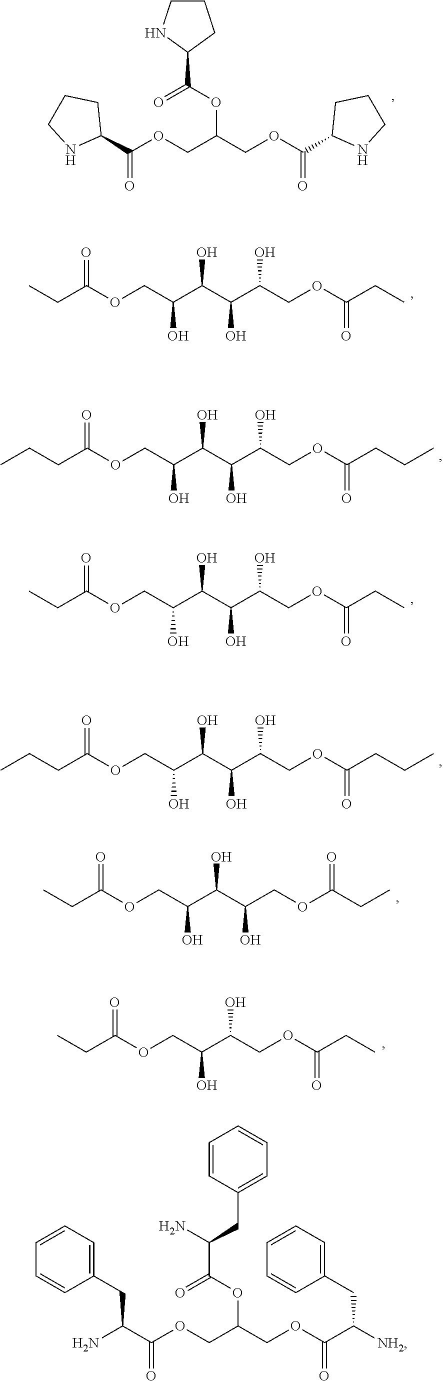

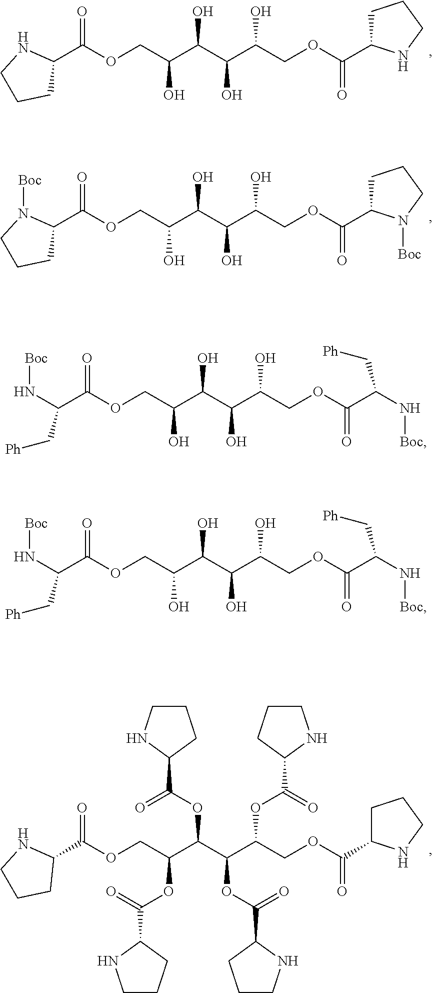

or salt thereof.

Various monosaccharides may be used to form an esterified polyol of the present disclosure. A monosaccharide may be a hexose. The hexose may be an aldohexose or a ketohexose. The hexose may be D-glucose, D-mannose, or D-fructose.

An esterified polyol made from a monosaccharide may have the following structure:

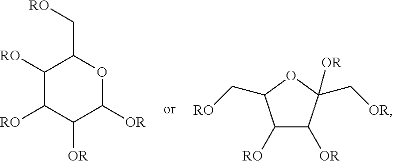

where each R is independently H or

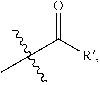

where each R′ is independently chosen from aliphatic groups, aryl groups, and amino acid groups (e.g.,

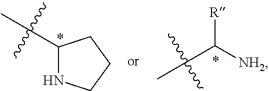

wherein the asterisked carbon has R stereochemistry or S stereochemistry and R″ is the sidechain of a canonical amino acid or the sidechain of a non-canonical amino acid) and at least one R is not H. The esterified polyol may have one or more stereogenic carbons (e.g., R or S).

Various aliphatic groups may be used. The aliphatic groups may be substituted or unsubstituted and/or linear or branched aliphatic groups. Non-limiting examples of aliphatic groups include a CH₃ group, a C₂H₅ group, linear and branched C₃H₇ groups, linear and branched C₄H₉, C₅H₁₁, and C₆H₁₃ groups, and the like.

Various aryl groups may be used. The aryl groups may be substituted or unsubstituted aryl groups. Non-limiting examples of aryl groups include phenyl groups;

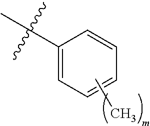

groups, where m is 1, 2, 3, 4, or 5; and the like.

Amino acid groups are formed from amino acids (e.g.,

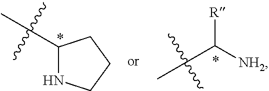

where the asterisked carbon has R stereochemistry or S stereochemistry and R″ is the sidechain of a canonical amino acid or the sidechain of a non-canonical amino acid). The amino acids may be canonical amino acids or non-canonical amino acids. The amino acids may be derivatives of glycine (e.g., a glycine functionalized at the amine). Examples of amino acid groups include, but are not limited to, prolinyl groups

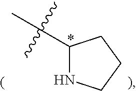 [SMILES: *C1CCCN1]

glycine betainyl
groups

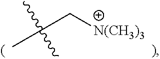

glycocyaminyl groups

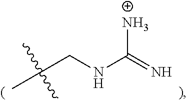 [SMILES: *CNC(=N)[NH3+]]

and the like.

An esterified polyol formed from a monosaccharide may have the following structure:

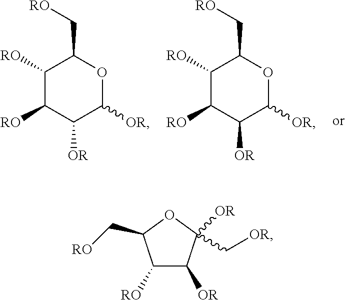

where none of the R groups are H and at least one R is a prolinyl group

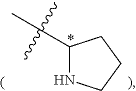 [SMILES: *C1CCCN1]

a glycine betainyl group

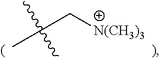

or a glycocyaminyl group

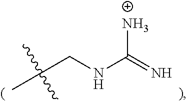 [SMILES: *CNC(=N)[NH3+]]

Various disaccharides may be used to form an esterified polyol of the present disclosure. The disaccharides may be combinations of various saccharides (e.g., pentoses and/or hexoses). In various examples, the disaccharide is sucrose or trehalose.

An esterified polyol made from a disaccharide may have the following structure:

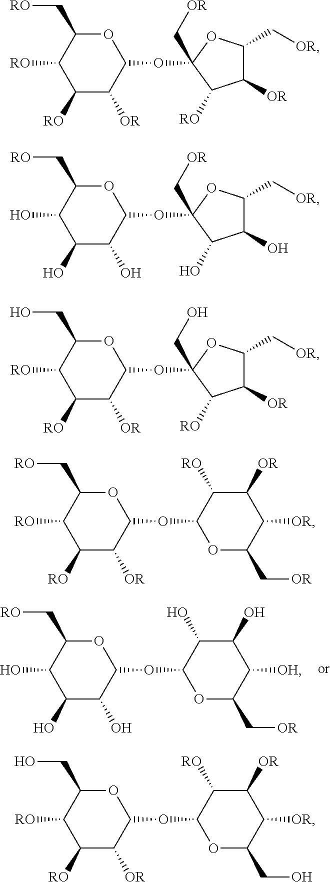

where each R is independently H or,

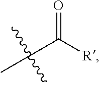

where each R′ is independently chosen from aliphatic groups, aryl groups, and amino acid groups (e.g.,

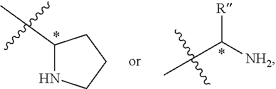

wherein the asterisked carbon has R stereochemistry or S stereochemistry and R″ is the sidechain of a canonical amino acid or the sidechain of a non-canonical amino acid) and at least one R is not H.

Various aliphatic groups may be used. The aliphatic groups may be substituted or unsubstituted aliphatic groups. Non-limiting examples of aliphatic groups include a CH₃ group, a C₂H₅ group, linear and branched C₃H₇ groups, linear and branched C₄H₉, C₅H₁₁, and C₆H₁₃ groups, and the like.

Various aryl groups may be used. The aryl groups may be substituted or unsubstituted aryl groups. Non-limiting examples of aryl groups include phenyl groups;

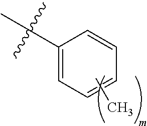

groups, where m is 1, 2, 3, 4, or 5; and the like.
Amino acid groups are formed from amino acids (e.g.,

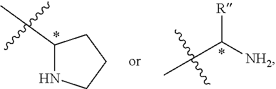

where the asterisked carbon has R stereochemistry or S stereochemistry and R″ is the sidechain of a canonical amino acid or the sidechain of a non-canonical amino acid). The amino acids may be canonical amino acids or non-canonical amino acids. The amino acids may be derivatives of glycine (e.g., a glycine functionalized at the amine). Examples of amino acid groups

include, but are not limited to, prolinyl groups

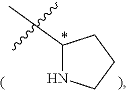 [SMILES: *C1CCCN1]

glycine betainyl groups

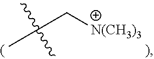

glycocyaminyl groups

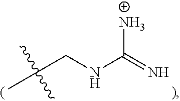 [SMILES: *CNC(=N)[NH3+]]

and the like.

In various examples, the esterified polyol of the present disclosure has the

following structure:

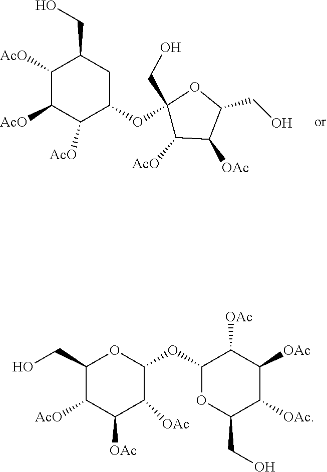 [SMILES: CC(=O)O[C@H]1[C@H](OC(C)=O)[C@@H](CO)C[C@H](O[C@]2(CO)O[C@H](CO)[C@@H](OC(C)=O)[C@@H]2OC(C)=O)[C@@H]1OC(C)=O ; CC(=O)O[C@@H]1[C@@H](OC(C)=O)[C@@H](O[C@H]2O[C@H](CO)[C@@H](OC(C)=O)[C@H](OC(C)=O)[C@H]2OC(C)=O)O[C@H](CO)[C@H]1OC(C)=O]

In an aspect, the present disclosure provides compositions comprising one or more esterified polyols of the present disclosure. The compositions further comprise one or more pharmaceutically acceptable carriers.

A composition may comprise additional components. For example, the composition comprises a growth medium or culture medium or a buffered solution suitable use in growth medium or culture medium. The growth medium or culture medium may be used to support the growth of microorganisms or cells (e.g., mammalian cells such as, for example, those of a human or a non-human).

A composition may comprise one or more standard pharmaceutically acceptable carrier(s). Non-limiting examples of compositions include solutions, suspensions, and emulsions. Non-limiting examples of diluents include distilled water for injection, physiological saline, vegetable oil, alcohol, and the like, and combinations thereof. Further, injections may contain stabilizers, solubilizers, suspending agents, emulsifiers, soothing agents, buffers, preservatives, and the like. The composition may also be formulated into a sterile solid preparation, for example, by freeze-drying, and can be used after sterilized or dissolved in sterile injectable water or other sterile diluent(s) immediately before use. Non-limiting examples of pharmaceutically acceptable carriers can be found in: Remington: The Science and Practice of Pharmacy (2012) 22nd Edition, Philadelphia, PA. Lippincott Williams & Wilkins.

Acceptable carriers, excipients, or stabilizers are nontoxic to recipients at the dosages and concentrations employed, and include, but are not limited to, buffers such as, for example, phosphate, citrate, histidine and other organic acids; antioxidants including, but not limited to, ascorbic acid and methionine; preservatives (such as, for example, octadecyldimethylbenzyl ammonium chloride; hexamethonium chloride; benzalkonium chloride, benzethonium chloride; phenol, butyl or benzyl alcohol; alkyl parabens such as, for example, methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, polyethylene glycol (PEG) and the like. In an embodiment, the pharmaceutical composition may comprise buffer components and stabilizers, including, but not limited to, sucrose, polysorbate 20, NaCl, KCl, sodium acetate, sodium phosphate, arginine, lysine, trehalose, glycerol, and maltose.

The compositions may have various usages. For example, a composition may be used in a method of the present disclosure. For example, the composition may be used a cryopreservative or used in a method in preparation of cryopreservation. In various other examples, the composition may be used for cosmetic applications (e.g., skin care and treatments). For example, a composition suitable for skin care may be used as a moisturizer.

In an aspect, the present disclosure provides methods of using one or more esterified polyols of the present disclosure. The one or more esterified polyols may be used in method for preparing a cell population for cryopreservation or in a method of cryopreserving a cell population or in a skin care application.

A method for preparing a cell population may comprise contacting the cell population in a suspension with one or more esterified polyols of the present disclosure. A method for cryopreserving a cell population may comprise contacting the cell population in a suspension with one or more esterified polyols of the present disclosure and subsequently freezing the cell population. In various examples, a method for cryopreserving a cell population or for preparing a cell population for cryopreservation does not involve comprising contacting the cells will DMSO prior to, during, or after contacting the cells with one or more esterified polyols of the present disclosure.

Various amounts of one or more esterified polyols may be used in the suspension prior to freezing or in the suspension for cryopreservation. For example, a suspension prior to freezing or in the suspension for cryopreservation may have an esterified polyol concentration of 0.1 to 100 mM, including every 0.01 mM value and range therebetween. In various examples, the concentration is 1-50 mM, 1-20 mM, 1-10 mM, 1-5 mM, 0.1-50 mM, 0.1-10 mM, 0.1-5 mM, or 5-10 mM. In various examples, the concentration of esterified polyol in a suspension prior to freezing or in the suspension for cryopreservation is 5 mM or 10 mM.

Various cells types may be used in a method of the present disclosure. For example, the cells of the cell population may be mammalian cells. Non-limiting examples of cells include stem cells, dendritic cells, red blood cells, natural killer cells, and the like.

In various examples, the esterified polyols of the present disclosure offer similar or better cryoprotection than that of DMSO while exhibiting less cytotoxicity than DMSO.

A method for skin care use may comprise application of a composition of the present disclosure to an individual (e.g., a composition comprising a compound of the present disclosure). The application may be topical application directly to a selected area (e.g., an area in need of, for example, moisturization, such as, for example, hands, feet, or other area having dry skin) of the individual. By “individual” it is meant a human or non-human animal (e.g., cow, pig, mouse, rat, cat, dog, or other agricultural, pet, or service animal, and the like). The composition may be an emulsion, oil-in-water emulsion, cream, lotion, ointment, or solution, or the like. Other suitable composition formulations may be known in the art. In various examples, the composition further comprises one or more additional components, such as, for example, water, a chelating agent, a moisturizing agent, a preservative, and/or a thickening agent, or the like, or a combination thereof.

The steps of the method described in the various embodiments and examples disclosed herein are sufficient to carry out the methods of the present disclosure. Thus, in an embodiment, the method consists essentially of a combination of the steps of the methods disclosed herein. In another embodiment, the method consists of such steps.

The following Statements describe various embodiments of the present disclosure:

Statement 1. An esterified polyol, wherein at least one or more or all of the alcohol groups is esterified.

Statement 2. An esterified polyol according to Statement 1, wherein the esterified polyol formed from a polyol chosen from polyhydric alcohols (which may be referred to as “sugar alcohols”), monosaccharides, and disaccharides.

Statement 3. An esterified polyol according to Statement 2, wherein the polyhydric alcohol has the following structure:

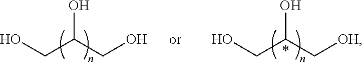

wherein n is 1, 2, 3, or 4 and the asterisked carbon has R or S stereochemistry or a racemate thereof.

Statement 4. An esterified polyol according any one of the preceding Statements, wherein the esterified polyol has the following structure:

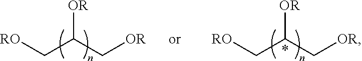

wherein n is 1, 2, 3, or 4 and each R is independently H or

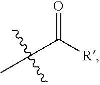

wherein each R′ is independently chosen from aliphatic groups, aryl groups, and amino acid groups (e.g.,

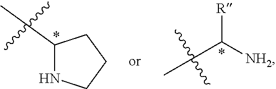

wherein the asterisked carbon has R stereochemistry or S stereochemistry and R″ is the sidechain of a canonical amino acid or the sidechain of a non-canonical amino acid) and at least one R is not H the asterisked carbon has R or S stereochemistry or a racemate thereof.

Statement 5. An esterified polyol according to claim 4, wherein each aliphatic group is chosen from a CH₃ group, a C₂H₅ group, linear and branched C₃H₇ groups, linear and branched C₄H₉, C₅H₁₁, and C₆H₁₃ groups, and the like.

Statement 6. An esterified polyol according to Statement 4, wherein the aryl group is a phenyl group; a

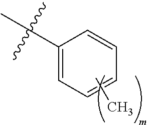

group, wherein m is 1, 2, 3, 4, or 5; or the like.

Statement 7. An esterified polyol according to Statement 4, wherein the amino acid group is a canonical amino acid group, a non-canonical amino acid group, or a group of a glycine-based derivative (e.g.,

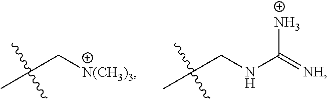

or the like).

Statement 8. An esterified polyol according to any one of Statements 4-7, wherein the esterified polyol is:

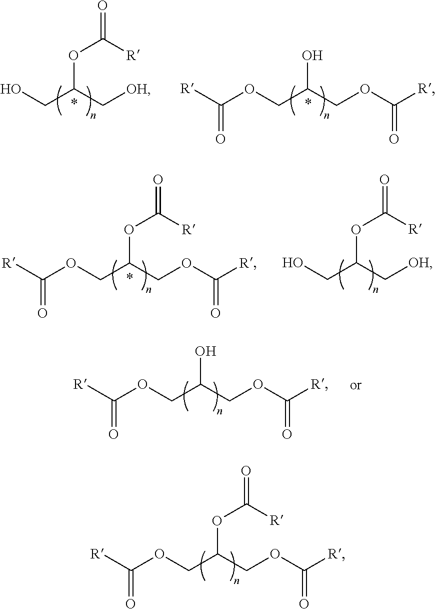

wherein the asterisked carbon has R or S stereochemistry or a racemate thereof.

Statement 9. An esterified polyol according to S, wherein the esterified polyol is:

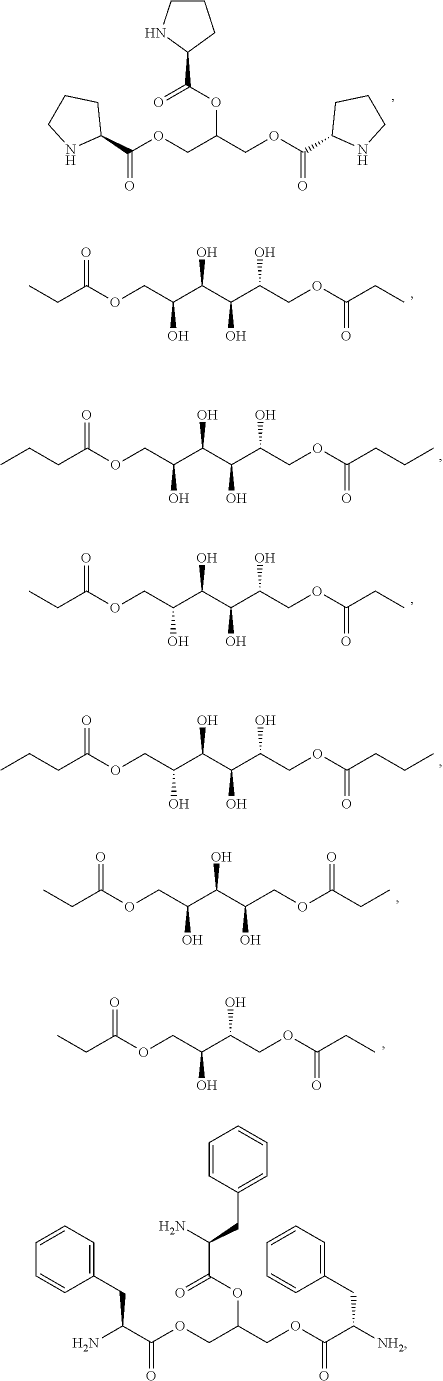

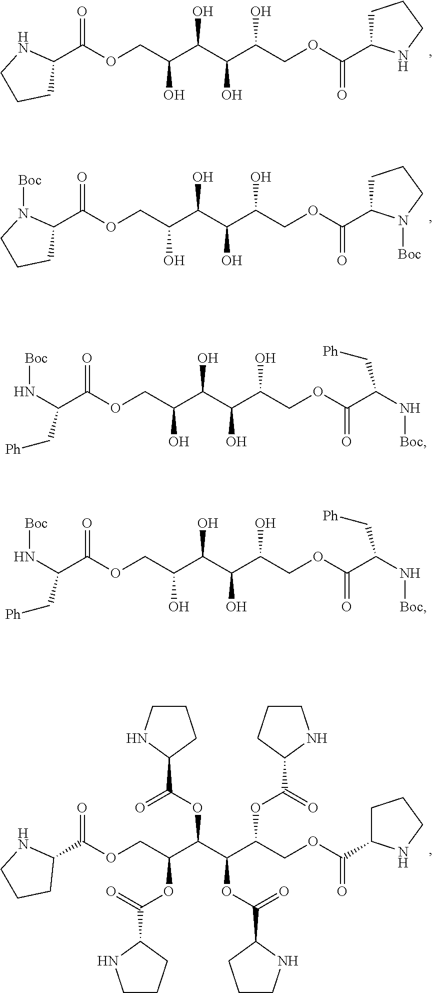

or salt thereof.

Statement 10. An esterified polyol according to Statement 2, wherein the monosaccharide is a hexose.

Statement 11. An esterified polyol according to Statement 10, wherein the hexose is D-glucose, D-mannose, or D-fructose.

Statement 12. An esterified polyol according to Statements 1, 2, 10, or 11, wherein the esterified polyol has the following structure:

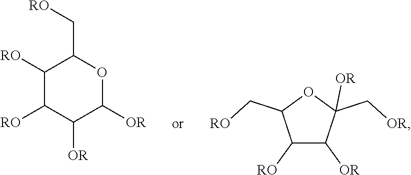

wherein each R is independently H or

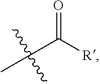

wherein each R′ is independently chosen from aliphatic groups, aryl groups, and amino acid groups (e.g.,

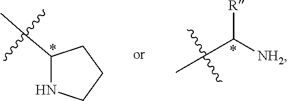

wherein the asterisked carbon has R stereochemistry or S stereochemistry and R″ is the sidechain of a canonical amino acid or the sidechain of a non-canonical amino acid) and at least one R is not H and one or more of the carbons have stereochemistry (R or S).

Statement 13. An esterified polyol according to Statement 12, wherein each aliphatic group is chosen from a CH₃ group, a C₂H₅ group, linear and branched C₃H₇ groups, linear and branched C₄H₉, C₅H₁₁, and C₆H₁₃ groups, and the like.

Statement 14. An esterified polyol according to Statement 12, wherein the aryl group is a phenyl group; a

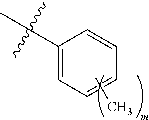

group, wherein m is 1, 2, 3, 4, or 5; or the like.

Statement 15. An esterified polyol according to Statement 12, wherein the amino acid group is a canonical amino acid group, a non-canonical amino acid group, or a group of a glycine-based derivative (e.g.,

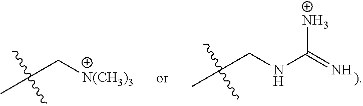

Statement 16. An esterified polyol according to Statements 10-15, wherein the esterified polyol is:

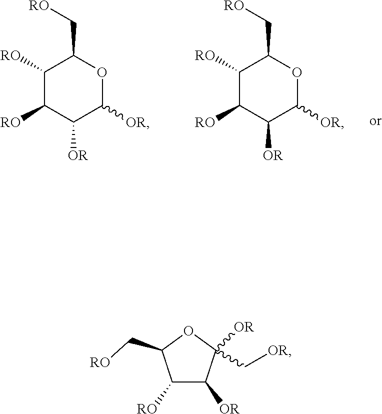

wherein none of the R groups are H.

Statement 17. An esterified polyol according to Statement 2, wherein the disaccharide is sucrose or trehalose.

Statement 18. An esterified polyol according to Statements 1, 2, or 17, wherein the esterified polyol has the following structure:

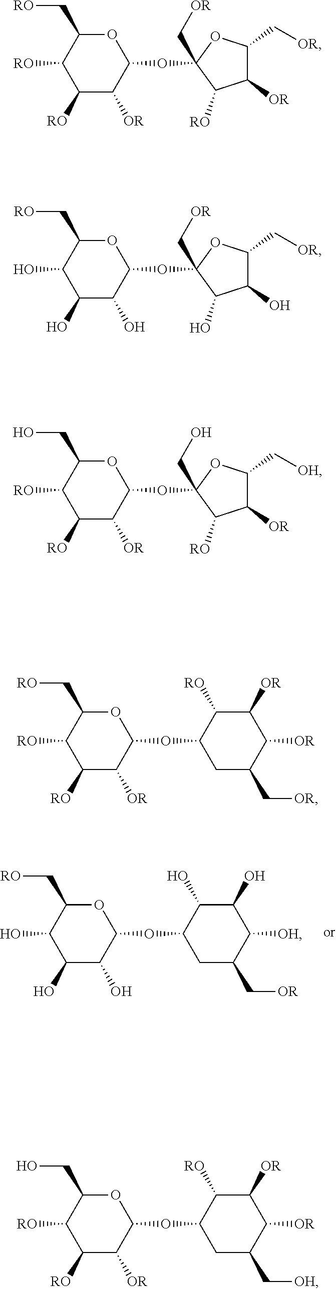

wherein R is

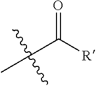

and each R′ is independently chosen from aliphatic groups, aryl groups, and amino acid groups (e.g.,

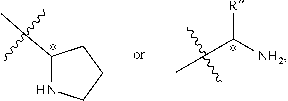

wherein the asterisked carbon has R stereochemistry or S stereochemistry and R″ is the sidechain of a canonical amino acid or the sidechain of a non-canonical amino acid).

Statement 19. An esterified polyol according to Statement 18 wherein each aliphatic group is chosen from a CH₃ group, a C₂H₅ group, linear and branched C₃H₇ groups, linear and branched C₄H₉, C₅H₁₁, and C₆H₁₃ groups, and the like.

Statement 20. An esterified polyol according to Statement 18, wherein the aryl group is a phenyl group; a group, wherein m is 1, 2, 3, 4, or 5; or the like.

Statement 21. An esterified polyol according to Statement 18, wherein the amino acid group is a canonical amino acid group, non-canonical amino acid group, or a group of a glycine-based derivative (e.g.,

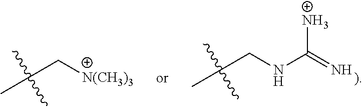

Statement 22. A composition comprising an esterified polyol according to any one of the preceding Statements.

Statement 23. A composition according to Statement 22, further comprising a buffered aqueous solution.

Statement 24. A method for preparing a cell population for cryopreservation comprising contacting the cell population in a suspension with an esterified polyol according to any one of Statements 1-21 or a composition according to Statement 22 or Statement 23, wherein the cell population is prepared for cryopreservation.

Statement 25. A method according to Statement 24, wherein the concentration of the esterified polyol in the suspension is 0.1 to 100 mM, including every 0.01 mM value and range therebetween.

Statement 26. A method according to Statement 25, wherein the concentration of the esterified polyol is 5 mM or 10 mM.

Statement 27. A method for cryopreserving a cell population comprising: contacting the cell population in a suspension with an esterified polyol according to any one of Statements 1-21 or a composition according to claim 22 or 23; freezing the suspension, wherein the cell population is cryopreserved.

Statement 28. A method according to Statement 27, wherein the concentration of the esterified polyol in the suspension is 0.1 to 100 mM, including every 0.01 mM value and range therebetween.

Statement 29. A method according to Statement 28, wherein the concentration of the esterified polyol is 5 mM or 10 mM.

Statement 30. A method for moisturizing skin comprising contacting a desired area of an individual with a composition comprising an esterified polyol of the present disclosure. For example, the esterified polyol may be:

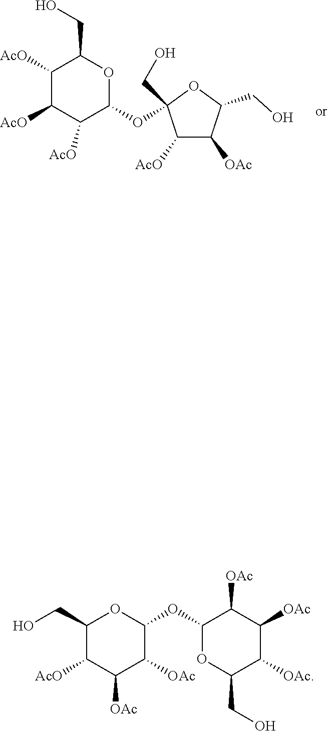 [SMILES: CC(=O)O[C@@H]1[C@@H](OC(C)=O)[C@@H](O[C@]2(CO)O[C@H](CO)[C@@H](OC(C)=O)[C@@H]2OC(C)=O)O[C@H](CO)[C@H]1OC(C)=O ; CC(=O)O[C@@H]1[C@H](OC(C)=O)[C@@H](O[C@H]2O[C@H](CO)[C@@H](OC(C)=O)[C@H](OC(C)=O)[C@H]2OC(C)=O)O[C@H](CO)[C@H]1OC(C)=O]

Statement 31. A method according to Statement 30, wherein the contacting comprises topically applying the composition.

Statement 32. A method according to Statement 30 or Statement 31, wherein the composition is an emulsion, an oil-in-water emulsion, cream, lotion, or a solution.

Statement 33. An esterified polyol, wherein the esterified polyol has the following structure:

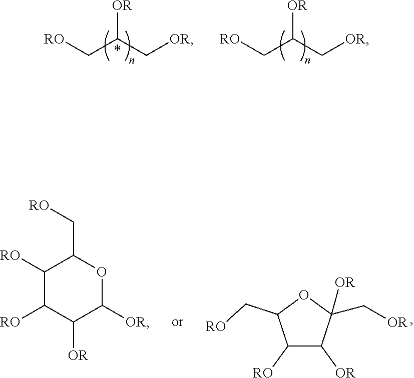

wherein n is 1, 2, 3, or 4 and each R is independently H or

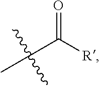

wherein each R′ is independently chosen from aliphatic groups, aryl groups, and amino acid groups and at least one R is not H, wherein the asterisked carbon has R stereochemistry or S stereochemistry, and when the esterified polyol has the following structure:

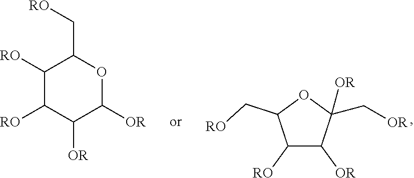

one or more of the carbons have R or S stereochemistry.

Statement 34. An esterified polyol according to Statement 33, wherein each aliphatic group is chosen from a CH₃ group, a C₂H₅ group, linear and branched C₃H₇ groups, linear and branched C₄H₉ groups, linear and branched C₅H₁₁ groups, and linear and branched C₆H₁₃ groups.

Statement 35. The esterified polyol according to Statement 33 or Statement 34, wherein the aryl group is a phenyl group or

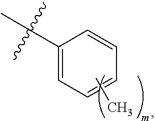

wherein m is 1, 2, 3, 4, or 5.

Statement 36. An esterified polyol according any one Statements 33-35, wherein the amino acid group is a canonical amino acid group, a non-canonical amino acid group, or an N-functionalized glycine-based derivative.

Statement 37. An esterified polyol according to any one of Statements 33-36, wherein the esterified polyol has the following structure:

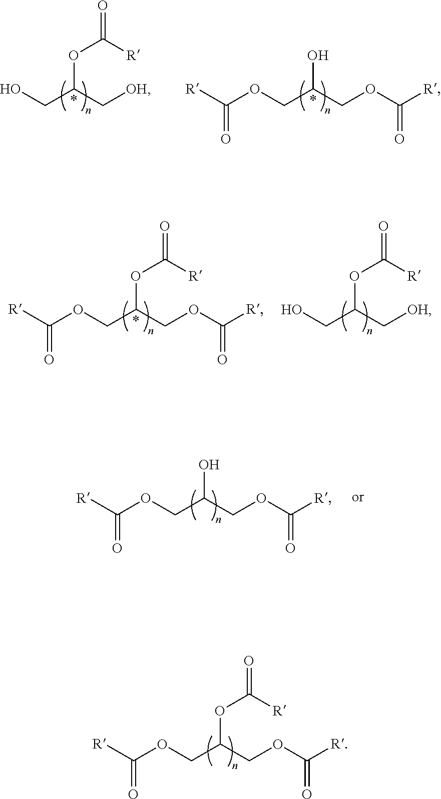

Statement 38. An esterified polyol according to any one of Statements 33-37, wherein the esterified polyol is:

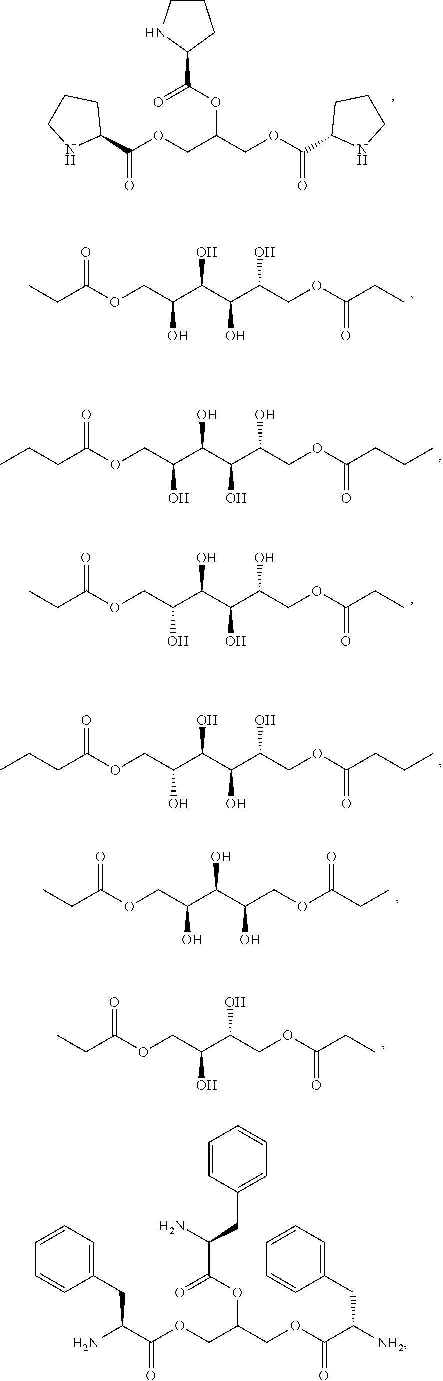

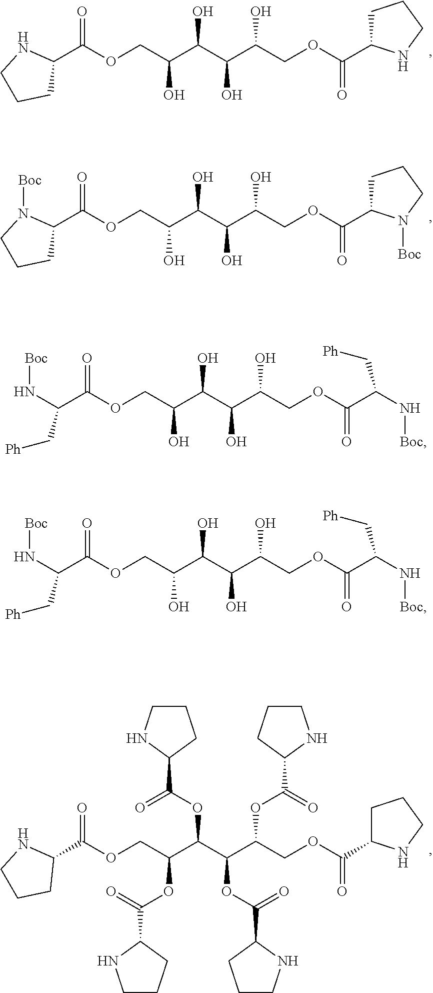

or salt thereof.

Statement 39. An esterified polyol according to any one of Statements 33-38, wherein the esterified polyol is:

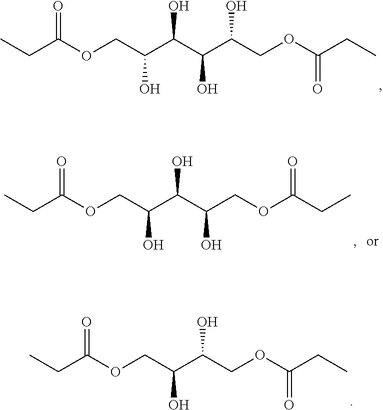

Statement 40. An esterified polyol according to Statement 33, wherein the esterified polyol is:

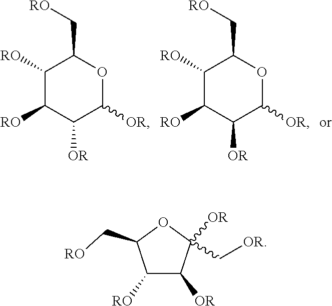

Statement 41. An esterified polyol having the following structure:

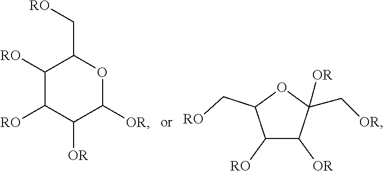

wherein n is 1, 2, 3, or 4 and each R is independently H, a saccharide group, or

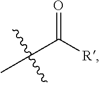

wherein each R′ is independently chosen from aliphatic groups, aryl groups, and amino acid groups and at least one R is not H and one or more of the carbons have R or S stereochemistry.

Statement 42. An esterified polyol according to claim 9, wherein the disaccharide is sucrose or trehalose.

Statement 43. An esterified polyol according to Statement 41 or Statement 42, wherein the esterified polyol has the following structure:

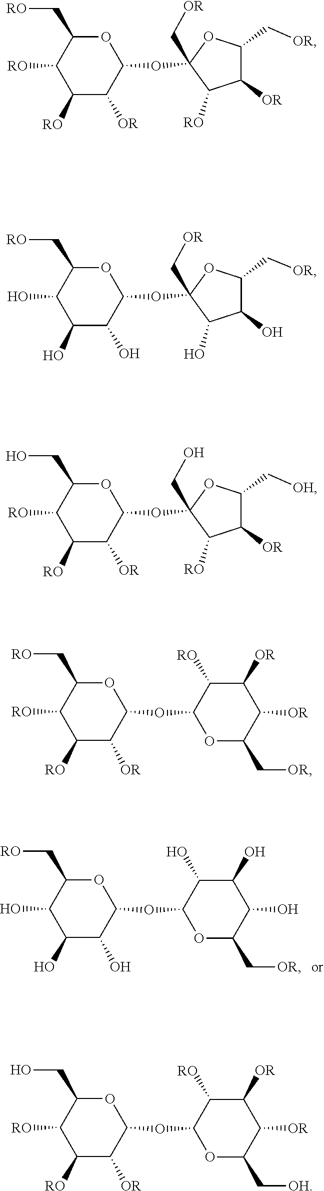

Statement 44. An esterified polyol according to any one of Statements 41-43, wherein each aliphatic group is chosen from a CH₃ group, a C₂H₅ group, linear and branched C₃H₇ groups, linear and branched C₄H₉ groups, linear and branched C₅H₁₁ groups, and linear and branched C₆H₁₃ groups.

Statement 45. An esterified polyol according to any one of Statements 41-44, wherein the aryl group is a phenyl group or

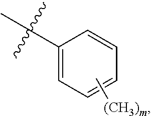

wherein m is 1, 2, 3, 4, or 5.

Statement 46. An esterified polyol according to any one of Statements 41-45, wherein the amino acid group is a canonical amino acid group, a non-canonical amino acid group, or an N-functionalized glycine-based derivative.

Statement 47. An esterified polyol according to any one of Statements 11, wherein the esterified polyol has the following structure:

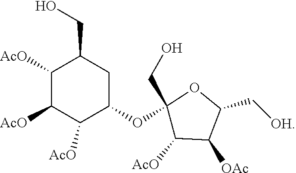 [SMILES: CC(=O)O[C@H]1[C@H](OC(C)=O)[C@@H](CO)C[C@H](O[C@]2(CO)O[C@H](CO)[C@@H](OC(C)=O)[C@@H]2OC(C)=O)[C@@H]1OC(C)=O]

Statement 48. A composition comprising an esterified polyol according to any one of Statements 33-47.

Statement 49. A composition according to Statement 48, further comprising a buffered aqueous solution.

Statement 50. A method for cryopreserving a cell population comprising: contacting the cell population in a suspension with an esterified polyol according to any one of Statements 33-47 or a composition according to any one of Statements 48-49, freezing the suspension, wherein the cell population is cryopreserved

Statement 51. The method according to Statement 50, wherein the concentration of the esterified polyol in the suspension is 0.1 to 100 mM (e.g., 5 mM or 10 mM).

Statement 52. A method for preparing a cell population for cryopreservation comprising contacting a population in a suspension with an esterified polyol according to any one of Statements 33-47 or a composition according to any one of Statements 48-49, wherein the cell population is prepared for cryopreservation.

Statement 53. A method according to Statement 52, wherein the concentration of the esterified polyol is in the suspension is 0.1 to 100 mM (e.g., 5 mM or 10 mM).

Statement 54. A method for moisturizing skin comprising contacting a desired area of an individual with a composition according to any one of Statements 48-49.

Statement 55. A method according to Statement 54, wherein the contacting comprises topically applying the composition.

Statement 56. The method according to Statement 54 or Statement 55, wherein the composition is an emulsion, oil-in-water emulsion, cream, lotion, or solution.

The following examples are presented to illustrate the present disclosure. They are not intended to be limiting in any matter.

### Example 1

This example provides a description of esterified polyols of the present disclosure and methods of using same.

Materials—Many sugars (monosaccharides and disaccharides) and sugar alcohols exhibit superior antifreeze properties but cannot penetrate cell membranes. As a result, these compounds can only be used as non-penetrating CPAs. This technology, by converting readily available sugar alcohols and sugars into their corresponding esters based on acylation with a biocompatible and nontoxic acids (i.e., RCOOH), provides water-soluble and membrane-permeable “pre-CPAs”. Upon entering cells, the internalized esters are hydrolyzed back into the original sugars or sugar alcohols, which function as potent CPAs inside cells, i.e., as penetrating CPAs.

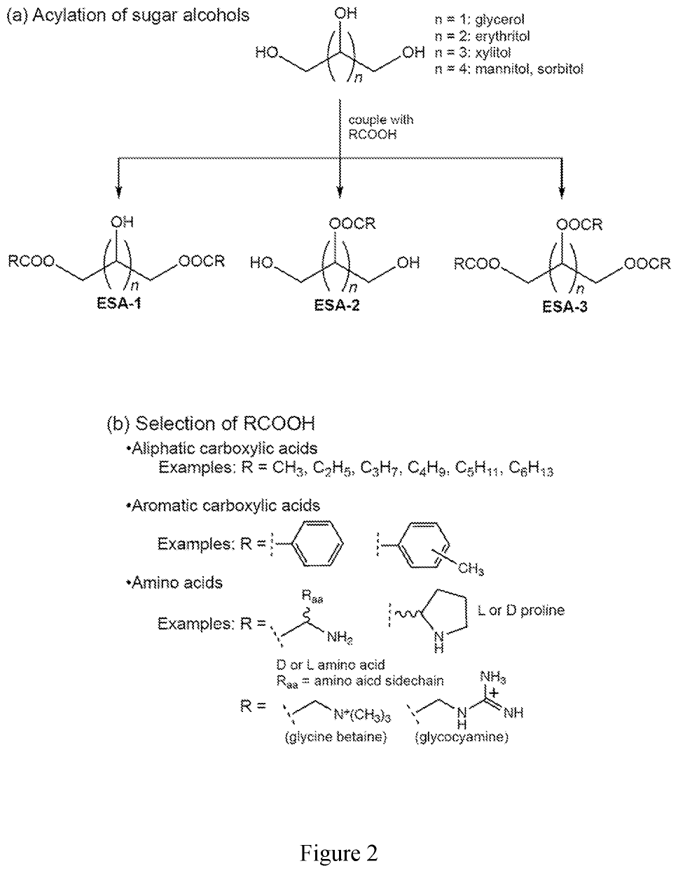

Derivatives of sugar alcohols. As shown in FIG. 2, five sugar alcohols with three to six carbons were acylated into three classes of esters ESA-1, ESA-2, and ESA-3.

Each of esters ESA-1 is derived from the acylation of the two primary hydroxyl groups of the corresponding sugar alcohol. Each of esters ESA-2 is derived from the acylation of the secondary hydroxyl groups of the corresponding sugar alcohol; each of esters ESA-3 is derived from the acylation of all the hydroxyl groups of the corresponding sugar alcohol. Aliphatic and aromatic carboxylic acids render the esters hydrophobic, which facilitates the membrane permeability; amino acids, glycine betain, and glycocyamine introduce multiple hydrophilic and cationic groups into the esters, which enhance solubility and also facilitate membrane penetration. Proline, glycine betaine, and glycocyamine are themselves antifreeze agents, which, upon being released intracellularly with sugar alcohol, further enhances the efficacies of the CPAs.

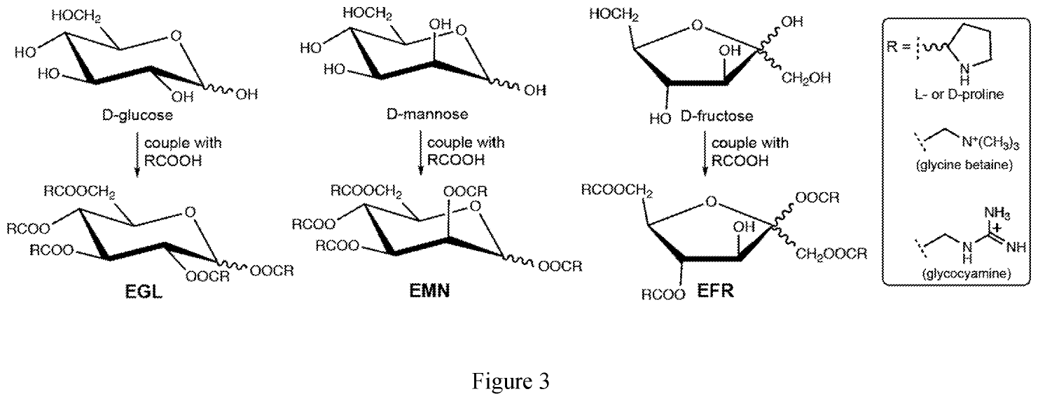

Modified monosaccharides. Glucose, mannose, and fructose, three readily available monosaccharides, were acylated with some of the acids (RCOOH) shown in FIG. 2 to give peracylated esters EGL, EMN, and EFR (FIG. 3). To prevent poor solubility, proline, glycine betaine and glycocyamine were used to acylate these monosaccharides. Esters EGL, EMN, and EFR are fully water-soluble and also membrane-permeable. Upon being hydrolyzed inside cells, the released proline, glycine betaine, or glycocyamine, serve as an intracellular (“penetrating”) CPA. The internalized monosaccharides may also serve as CPAs or can also be metabolized inside cells.

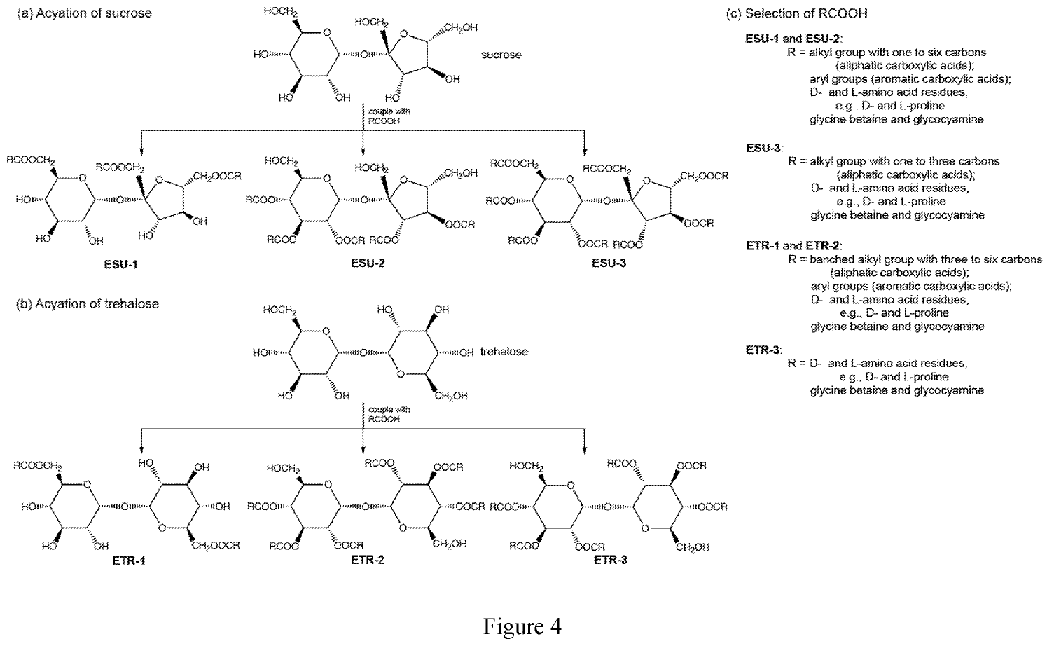

Modified disaccharides. Fructose and trehalose were modified to give esters corresponding to the acylation of the primary (ESU-1 and ETR-1), secondary (ESU-2 and ETR-2), and all (ESU-3 and ETR-3) hydroxyl groups (FIG. 4). The acids used for coupling with sucrose and trehalose to give each class of esters are shown in FIG. 4c.

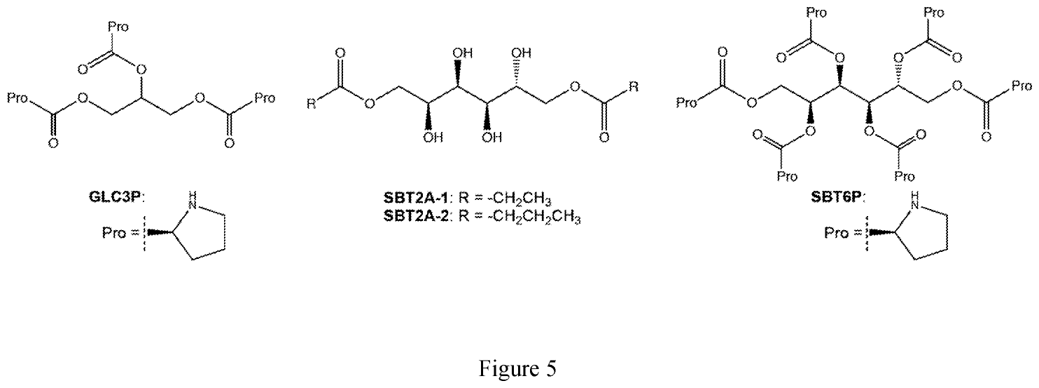

Cryopreservation of cells—The cryoprotective effects of four compounds derived from glycerol and sorbitol (FIG. 5) are examined. Ester GLC3P is derived from glycerol and L-proline, with the three hydroxyl groups of glycerol being acylated by the carboxyl group of L-proline; esters SBT2A-1 and SBT2A-2 are from acylating the primary hydroxyl groups of D-sorbitol with propionic acid and butanoic acid, respectively; ester SBT6P is the per-acylated ester of sorbitol with L-proline. These esters, with good solubilities in aqueous media, were applied to culture and freezing media from aqueous stock solutions without involving any organic solvent, leading to DMSO-free culturing and freezing conditions to be adopted. The obtained cryopreservation efficacies of these compounds demonstrate that this technology offers a protocol that allows the replacement of DMSO with non-toxic CPAs derived from readily available compounds and with cryopreservative outcome superior to that shown by DMSO.

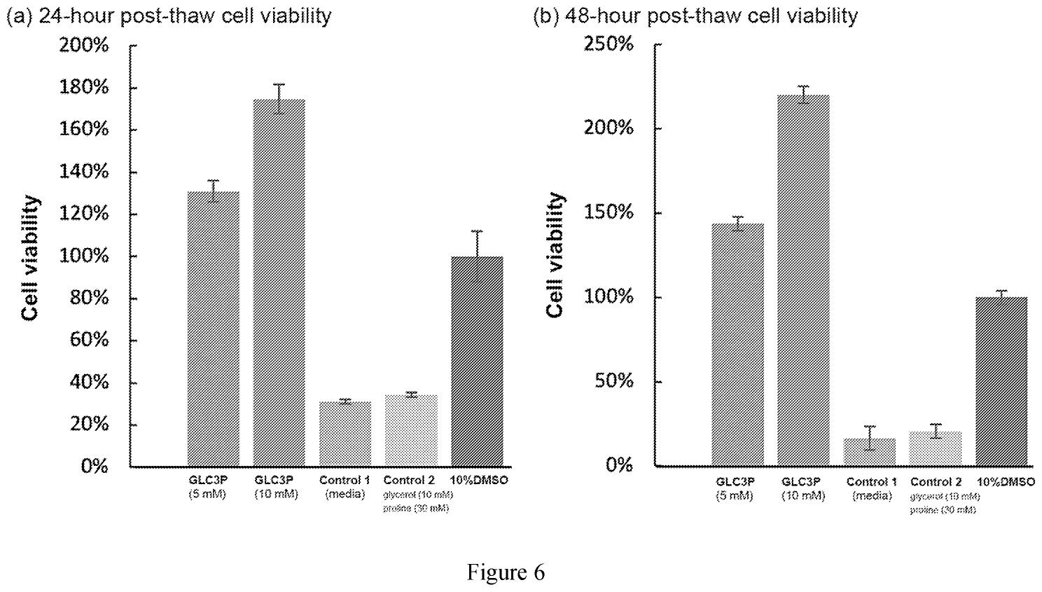

Post-thaw viability of NIH-3T3 cells cryopreserved with GLC3P. The viability of NIH-3T3 cells cryopreserved with GLC3P are shown in Table 1 and FIG. 6.

TABLE 1
Post-thaw viability of NIH-3T3 cells treated
with GLC3P (see FIG. 5 for structure).

| Cell Group^{a, b, c} | 24 hours post thaw | 48 hours post-thaw |
|---|---|---|
| Treated 1 | 130.9 ± 5.3 | 143.8 ± 4.2 |
| Treated 2 | 174.7 ± 6.9 | 220.4 ± 5.5 |
| Control 1 | 31.0 ± 1.1 | 16.4 ± 6.5 |
| Control 2 | 34.3 ± 1.1 | 20.5 ± 4.1 |
| DMSO | 100.0 ± 11.8 | 100.0 ± 4.0 |

^{a}(i) Cells of Treated groups 1 and 2 are incubated with GLC3P at 5 and 10 mM, respectively, without DMSO. (ii) Cells of Control groups 1 and 2 are incubated without GLC3P. Control 1 differs from Control 2 in that Control 2 contains 10 mM glycerol and 30 mM L-proline. Other conditions are identical to those of the treated groups. (iii) Cells of the DMSO group are incubated with 10% DMSO which replaces GLC3P while the other conditions are identical to those of the treated groups.
^{b}All cells were frozen at −80° C. for 72 hours and re-cultured for 24, and 48 hours.
^{c}Incubation media = 2 mL DMEM (pH 7.4) with 10% FBS; freezing media = DMEM (pH 7.4), 10% FBS, 400 mM trehalose.

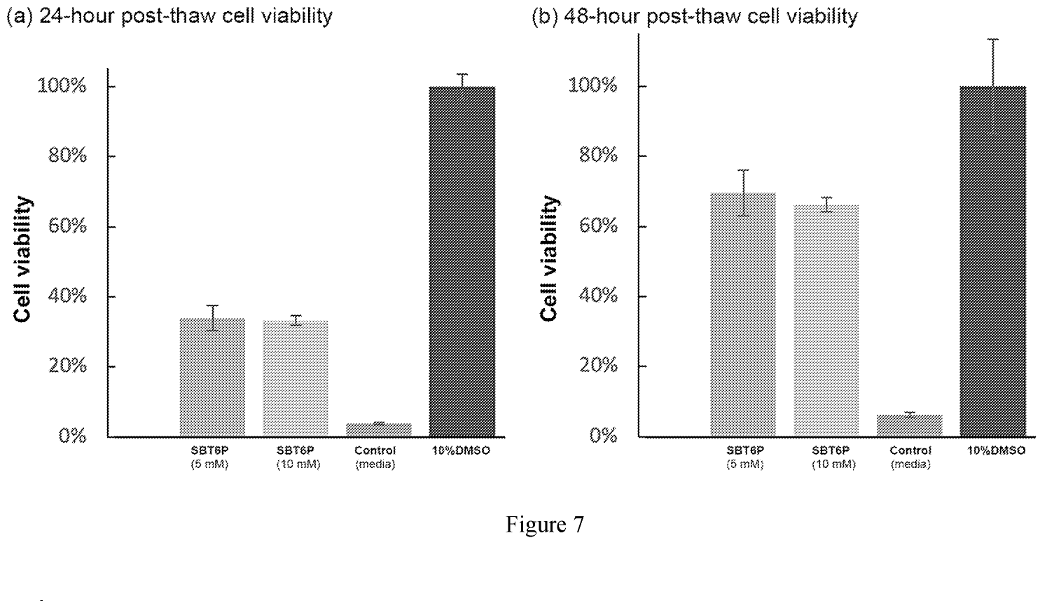

Post-thaw viability of NIH-3T3 cells cryopreserved with SBT6P. The viability of NIH-3T3 cells cryopreserved with SBT6P are shown in Table 2 and FIG. 7.

TABLE 2
Post-thaw viability (%) of NIH-3T3 cells treated
with SBT6P (see FIG. 5 for structure).

| Cell Group^{a, b, c} | 24 hours post thaw | 48 hours post-thaw |
|---|---|---|
| Treated 1 | 33.3 ± 1.4 | 66.2 ± 2.0 |
| Treated 2 | 34.0 ± 3.5 | 69.6 ± 6.5 |
| Control | 3.0 ± 0.3 | 6.3 ± 0.7 |
| DMSO | 100.0 ± 3.6 | 100.0 ± 13.4 |

^{a}(i) Cells of Treated groups 1 and 2 are incubated with SBT6P at 5 and 10 mM, respectively, without DMSO. (ii) Cells of Control group 1 are incubated in culture media without SBT6P. (iii) Cells of the DMSO group are incubated with 10% DMSO which replaces SBT6P while the other conditions are identical to those of the treated groups.
^{b}All cells are incubated for 36 hours before being frozen at −80° C. for 72 hours and re-cultured for 24, and 48 hours.
^{c}Incubation media = 2 mL DMEM (pH 7.4) with 10% FBS; freezing media = DMEM (pH 7.4), 10% FBS, 400 mM trehalose.

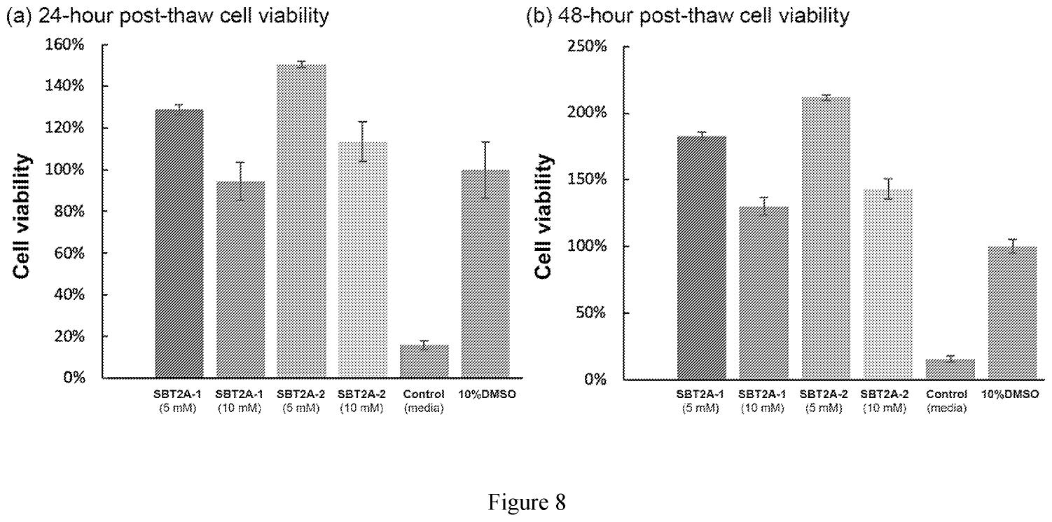

Post-thaw viability of NIH-3T3 cells cryopreserved with SBT2A-1 and SBT2A-2. The viability of NIH-3T3 cells cryopreserved with SBT2A-1 and SBT2A-2 are shown in Table 3 and FIG. 8.

TABLE 3
Post-thaw viability (%) of NIH-3T3 cells treated with
SBT2A-1 and SBT2A-2 (see FIG. 5 for structure).

| Cell Group^{a, b, c} | 24 hours post thaw | 48 hours post-thaw |
|---|---|---|
| Treated 1 | 94.5 ± 9.1 | 130.1 ± 6.8 |
| Treated 1a | 128.9 ± 2.5 | 183.0 ± 2.9 |
| Treated 2 | 113.4 ± 9.5 | 143.3 ± 7.4 |
| Treated 2a | 150.4 ± 1.5 | 211.8 ± 2.2 |
| Control | 15.8 ± 2.2 | 15.6 ± 2.2 |
| DMSO | 100.0 ± 13.5 | 100.0 ± 5.3 |

^{a}Cells of Treated groups 1 and la are incubated with SBT2A-1 at 5 and 10 mM, respectively; and cells Treated groups 2 and 2a are incubated with SBT2A-2 at 5 and 10 mM, respectively, without DMSO. (ii) Cells of Control group are incubated in culture media without SBT2A-1 or SBT2A-2. (iii) Cells of the DMSO group are incubated with 10% DMSO which replaces SBT2A-1 or SBT2A-2 while the other conditions are identical to those of the treated groups.
^{b}All cells are incubated for 36 hours before being frozen at −80° C. for 72 hours and re-cultured for 24, and 48 hours.
^{c}Incubation media = 2 mL DMEM (pH 7.4) with 10% FBS; freezing media = DMEM (pH 7.4), 10% FBS, 400 mM trehalose.

Methods—Synthesis. The synthetic modifications of a sugar or sugar alcohol involved the acylation of the (1) primary, (2) secondary, or (3) all hydroxyl groups.

The general procedures, shown in FIG. 1, for modifying sugars and sugar alcohols are exemplified with the acylation of sorbitol and sucrose. The synthetic steps started with first blocking the primary hydroxyl groups of sorbitol or sucrose with tritylation, followed by acylation of the secondary hydroxyl groups to give the tritylated tetra- or penta-ester. Removing the trityl groups gave the tetra-ester SBT4A or penta-ester ESU-2. The secondary hydroxyl groups of the tritylated sorbitol or sucrose were blocked with benzyl groups, followed by removing the trityl groups and acylation of the primary hydroxyl groups, and then removal of the benzyl groups to give ester SBT2A or ESU-1. Finally, the per-acylated esters SBT6A or ESU-3 were obtained by coupling sorbitol or sucrose with the corresponding acid, acid chloride/anhydride. For amino acid such as proline, the N-CBZ or N-Boc protected form were used in the coupling steps, followed by removal the CBZ or Boc group. Other sugars and sugars alcohols were similarly modified.

Cryopreservation protocol. The procedure described below was typically repeated three times to collect statistically meaningful data.
Cultured NIH-3T3 cells in 6-well plate for 24 hours first to allow the cells to attach to the plate (5×10⁴ cells in 2 mL DMEM with 10% FBS per well).
Replaced the media (DMEM (pH=7.4) with 10% FBS for control group, 5 mM or 10 mM of the ester of a sugar or sugar alcohol in DMEM (pH=7.4) with 10% FBS (filtered with 0.22 μm filter) for treated group).
Incubated the cells for 72 hours.
Discarded the media, washed each well with 1 mL 1× PBS (pH=7.4) and treated each well with 1 mL 0.25% trypsin-EDTA (incubating for 10-20 minutes) to detach the cells.
Added another 1 mL DMEM (pH=7.4) with 10% FBS to quench the trypsin, centrifuge and collected the cell as pellet. Discarded 95% of the old media and added 0.5 mL new DMEM (pH=7.4) with 10% FBS and 400 mM trehalose (filtered with 0.22 μm filter) and re-suspended the cells.
Transferred the cell culture to cryo-tube and stored the tubes at −80° C. for 3 days.
Thawed the cells at 37° C. in a water bath until ice melted.
Counted the cell density in each cryo-tube with cell counter.
Re-cultured the cells in 96-well plate with fresh DMEM (PH=7.4) with 10% FBS. (while keeping cell density the approximately the same for each well).
Used CCK-8 kit to test the post-thaw viability after certain time.

### Example 2

This example provides a description of esterified polyols of the present disclosure and methods of using same.

Sorbitol, mannitol, xylitol, and erythritol, four readily available sugar alcohols with poor or no membrane permeability, were converted into their corresponding dipropionates by acylating their primary hydroxyl groups. With enhanced membrane-permeability, these diesters are expected to permeate the cell membranes and upon their hydrolysis, release the corresponding sugar alcohols inside the cells. NIH-3T3 cells incubated with these diesters before being frozen at −80° C. exhibited considerably higher total recovery over those incubated with the free sugar alcohols or media only. Among the four diesters, those of sorbitol, especially mannitol, showed cryoprotective effects comparable to that shown by 5% DMSO. This work has demonstrated the feasibility of converting readily available, naturally occurring compounds into membrane-permeable derivatives that serve as water-soluble, non-toxic alternatives to DMSO.

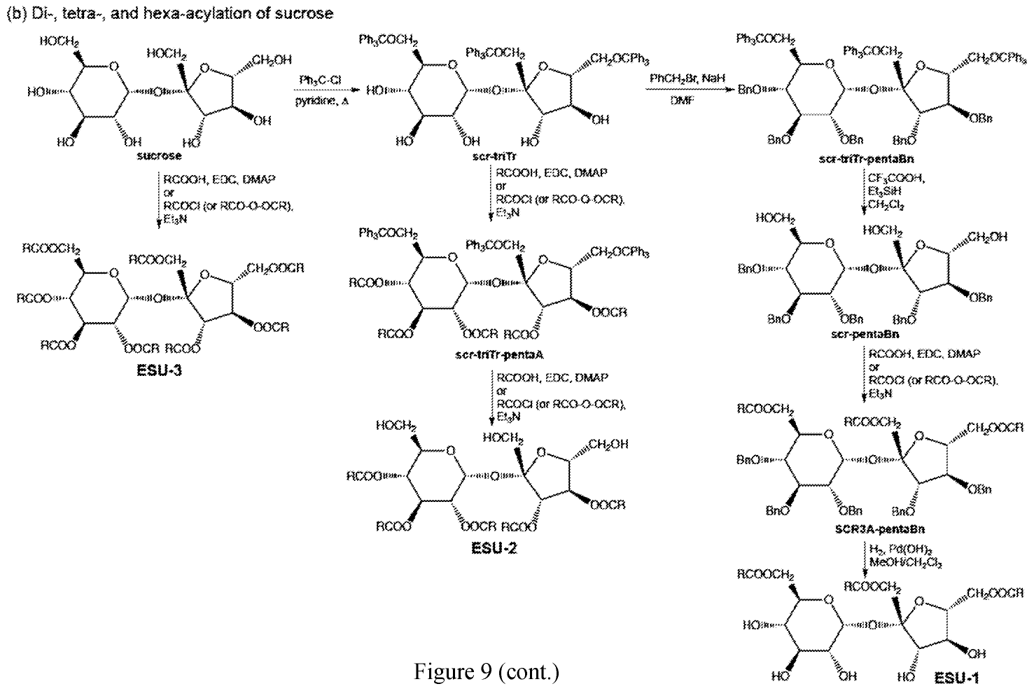

Described herein is the identification of effective CPAs derived from the partial acylation of sorbitol, mannitol, xylitol, and erythritol, four readily available sugar alcohols. FIG. 10 shows the five-step synthesis of six diesters which include the dipropionates (1-4) of sorbitol, mannitol, xylitol, and erythritol, and the dibutyrates of sorbitol (1a) and mannitol (2a) (see also Scheme 1). The six diesters were obtained in good yields and were fully characterized with multiple analytical techniques with satisfactory results. With two to four remaining free hydroxyl groups, the diesters retain good aqueous solubilities. With their propionyl or butyric groups, each of the diesters gains enhanced hydrophobicity and is expected to be more membrane-permeable than the corresponding sugar alcohol, which should facilitate its intracellular delivery.

By examining the cryoprotection of cells with the six diesters, the following questions were addressed: (1) Compared to unmodified sugar alcohols, can the four esters lead to improved cryoprotective outcomes including enhanced post-thaw cell viability and growth? (2) If good cell protection can indeed be realized with some or all of the four esters, are the protective effects comparable to that shown by DMSO? (3) Do different esters show the same or different cryoprotective effectiveness?

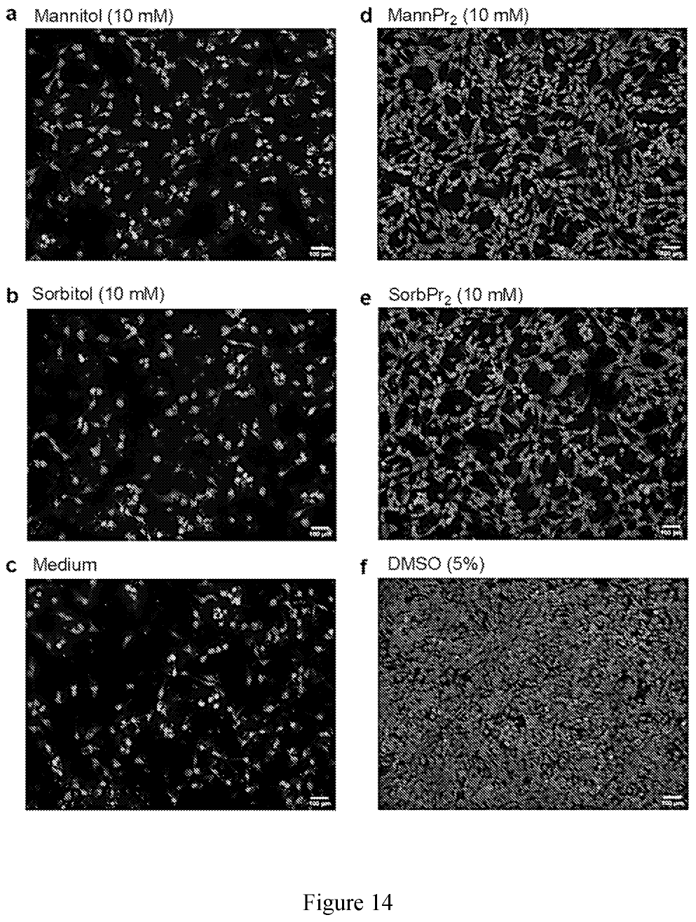

Cytotoxicity assays were performed by exposing NIH-3T3 cells to the diesters. Cell viability was determined by using Cell Counting Kit-8 (CCK-8) reagent and a BioTek microplate reader to count the numbers of live and dead cells. None of the four dipropionates showed any apparent toxicity to the cells, with cell viability of ˜70% to 100% relative to that of the control groups being observed in the presence of up to 10 mM of each diester (FIG. 15). In contrast, the two dibutyrates, SorbBa₂ (1a) and MannBa₂ (2a), exhibited noticeable cytotoxicity, with cell viability of less than 40% of that of the control groups being observed at 10 mM.

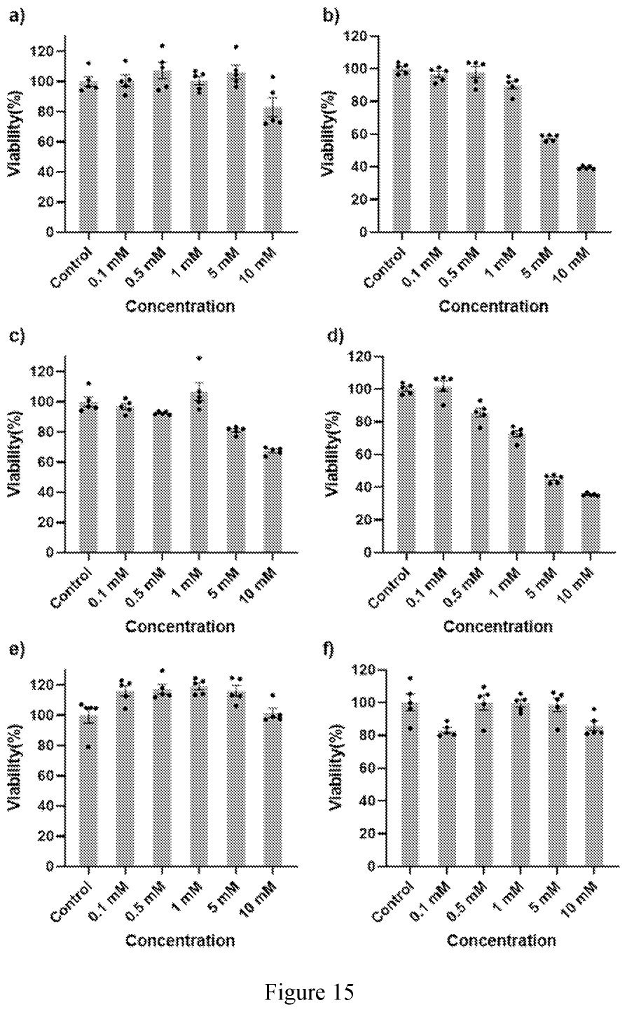

Propionic acid and butyric acid, which were released upon the hydrolysis of the diesters inside cells, were examined for their toxicity to NIH-3T3 cells. The obtained cell viability reveals insignificant cytotoxicity for propionic acid, with cell viability remaining at ˜70% at up to 10 mM (FIG. 16). In contrast, cell viability dropped to ˜50% and ˜40% in the presence of 5 mM and 10 mM, respectively, of butyric acid. Thus, the observed suppression of cell growth by SorbBa₂ and MannBa₂ seems to be caused by butyric acid released upon the hydrolysis of the two dibutyrates. The subsequent cryoprotection studies were thus based on dipropionates 1-4.

After being internalized, a diester either directly serves as a CPA, or more likely, is cleaved enzymatically by nonspecific esterases or non-enzymatically due to background hydrolysis of the ester groups. The conversion of a diester into a membrane-impermeable sugar alcohol inside cells shifted the equilibrium across the cell membrane, leading to the accumulation of the sugar alcohol inside the cells to concentration(s) higher than the initially added concentration of the diester. The internalized sugar alcohol, perhaps along with any unhydrolyzed diester that remains inside the cells, acts as an intracellular CPA.

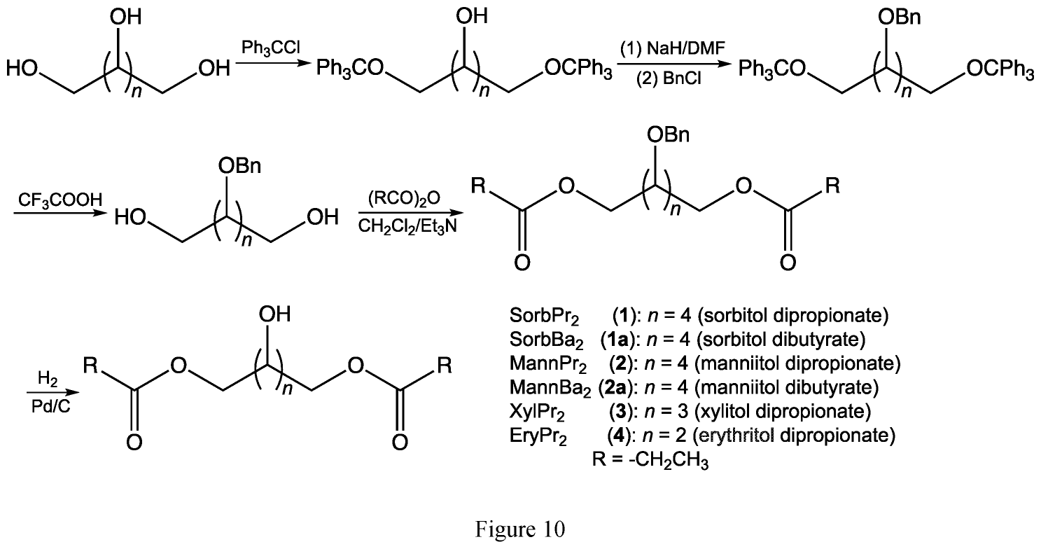

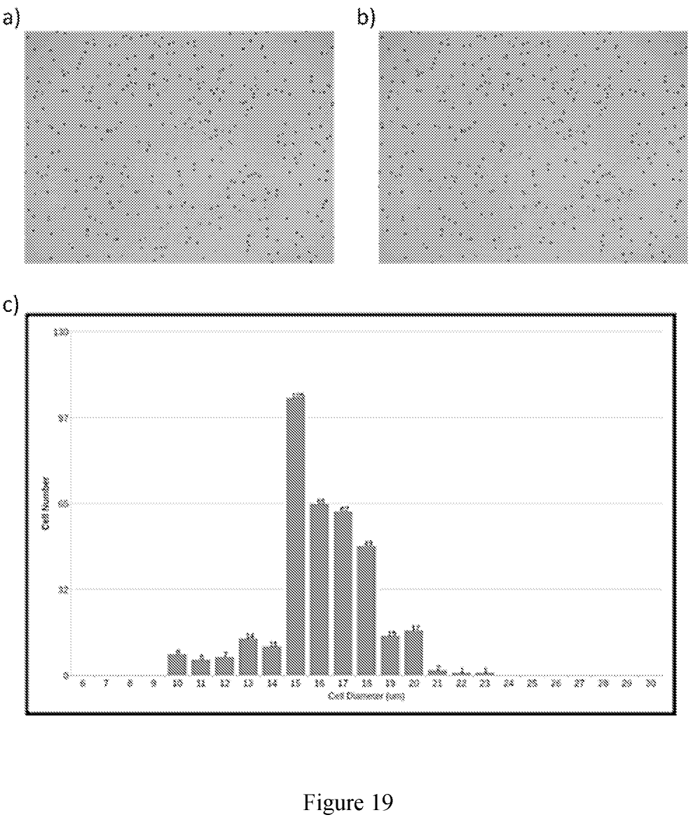

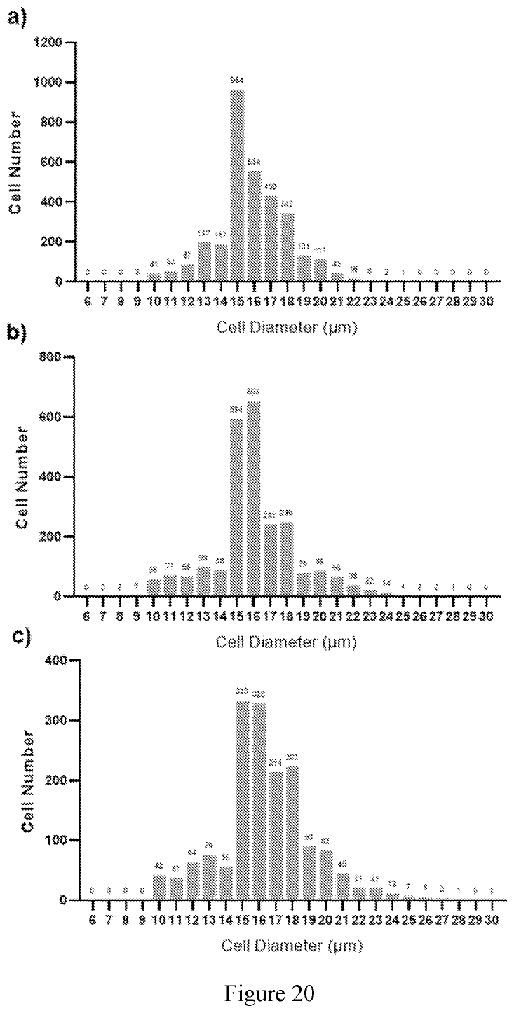

The sizes of NIH-3T3 cells before and after being incubated with diesters SorbPr₂ and MannPr₂ were examined. As shown in FIG. 11, cells incubated with MannPr₂ and SorbPr₂ for 48 h underwent swelling, as indicated by their average diameters which show small but statistically significant increases relative to the size of cells incubated with only the culture medium (see FIGS. 19 and 20). The observed size increase provides qualitative evidence that is consistent with the expected membrane-permeability of the diesters, which allows their uptake by the cells. The hydrolysis of SorbPr₂ or MannPr₂ inside the cells releases sorbitol or mannitol. As impermeant solutes, the sugar alcohols get accumulated and create osmotic pressure gradients that drive the influx of water molecules, resulting in the swelling of the cells.

The delivery of sugar alcohols into cells makes it possible to turn these molecules into intracellular CPAs. NIH-3T3 cells were employed to assessed the cryopreservation effects of dipropionates 1a-d because of the ready availability and relatively short doubling time (˜20 h) of these cells, as well as the demonstrated use of this cell line in previously reported studies of cryopreservation. Total post-thaw cell recovery, defined as the ratio (%) of post-thaw live cells to the number of initially frozen live cells, which provides a more accurate measure of cryoprotective outcome than cell viability, was used to assess the cryoprotective effects of the diesters.

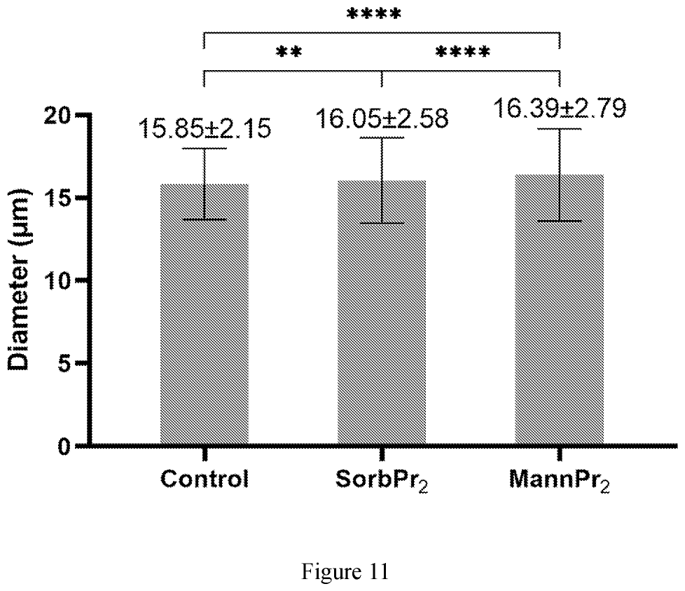

In Gibco Dulbecco's Modified Eagle Medium (DMEM) containing 10% fetal bovine serum (FBS), NIH-3T3 cells were incubated with 5 and 10 mM of SorbPr₂ to assess the effect of concentration on cell cryoprotection. As shown in FIG. 12, at both 5 and 10 mM, the post-thaw 0 h and 48 h total recovery rates of cells incubated with SorbPr₂ were above those of cells incubated only with the media, which indicates that SorbPr₂ is able to protect NIH-3T3 cells, with the protection being more effective at 10 mM than 5 mM.

Systematic studies were then performed by incubating the cells (1) with each of diesters 1-4 (10 mM), (2) with unmodified sorbitol, mannitol, xylitol, or erythritol (10 mM), and (3) in medium without any of the diesters or sugar alcohols, at 37° C. for 48 h. The cells treated with diesters 1-4 and the four sugar alcohols were then transferred to freezing media consisting of DMEM, 10% FBS and 400 mM trehalose. The cells incubated with only the medium were divided into two portions, one being directly transferred to the freezing medium containing 400 mM trehalose (control 1) and the other being placed in a different freezing medium consisting of DMEM, 10% FBS and 5% DMSO but without trehalose (control 2). After being frozen at −80° C. for 72 h, the cells were thawed in a warm water bath at 37° C. until ice melted. The post-thaw cells were either immediately analyzed for their survival or were cultured in growth media (DMEM with 10% FBS) for 24 h and 48 h before being analyzed.

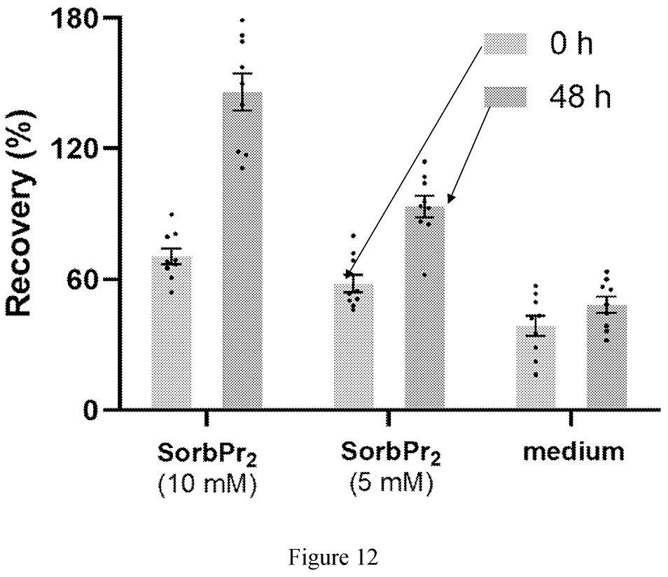

The total recovery values of NIH-3T3 cells, obtained immediately (0 h), 24 h, and 48 h post-thaw, are shown in FIG. 13 and Table 4, which allow not only the immediate post-thaw survival but also the growth and continued health of the cryopreserved cells to be assessed.

The post-thaw total recovery values of cells incubated with the four free sugar alcohols, ranging between 20-30%, are similar or lower than those of cells cultured with media only (control-1), indicating that the free alcohols, being membrane-impermeable and unable to enter the cells, offer negligible cryoprotection for NIH-3T3 cells. In contrast, the post-thaw total recovery values of the cells incubated with the diesters range from the lowest 48% (0 h) and 72% (48 h) with EryPr₂ to the highest 84% (0 h) and 184% (48 h) with MannPr₂, which are considerably higher than the recovery rates of cells treated with the free sugar alcohols or media only. Thus, compared with the corresponding sugar alcohols, the diesters can indeed better protect cells from freezing and thawing damages during cryopreservation. The internalized free sugar alcohols and, perhaps along with unhydrolyzed diesters, if any, that remain inside the cells, act as intracellular CPAs which result in the observed increase in cell viability.

The different protective outcomes observed with the four dipropionates indicate that these compounds, although being at the same concentration (10 mM), differentially impact the survival of cells that experienced the freezing and thawing processes, i.e., these molecules do not function via a concentration-dependent (colligative) mechanism. Instead, the observed different cryoprotective effects were most likely due to the different capabilities of the sugar alcohols, which are generated intracellularly from the hydrolysis of the diesters, in reducing ice content or protecting macromolecules such as proteins and membranes.

The much higher total recovery rates of cells treated with MannPr₂ and SorbPr₂ than those of cells treated with XylPr₂ and EryPr₂ and the different protective effects of MannPr₂ and SorbPr₂ further indicate that the observed cryoprotective outcomes are the results of the specific identities of the involved molecules. Mannitol and sorbitol, two isomers sharing the same number and distribution of hydroxyl groups, seem to be very similar. With different chirality i.e., the orientations of the hydroxyl groups in three-dimensional space, mannitol and sorbitol probably engages in different interactions with ice nuclei and/or biomacromolecules.

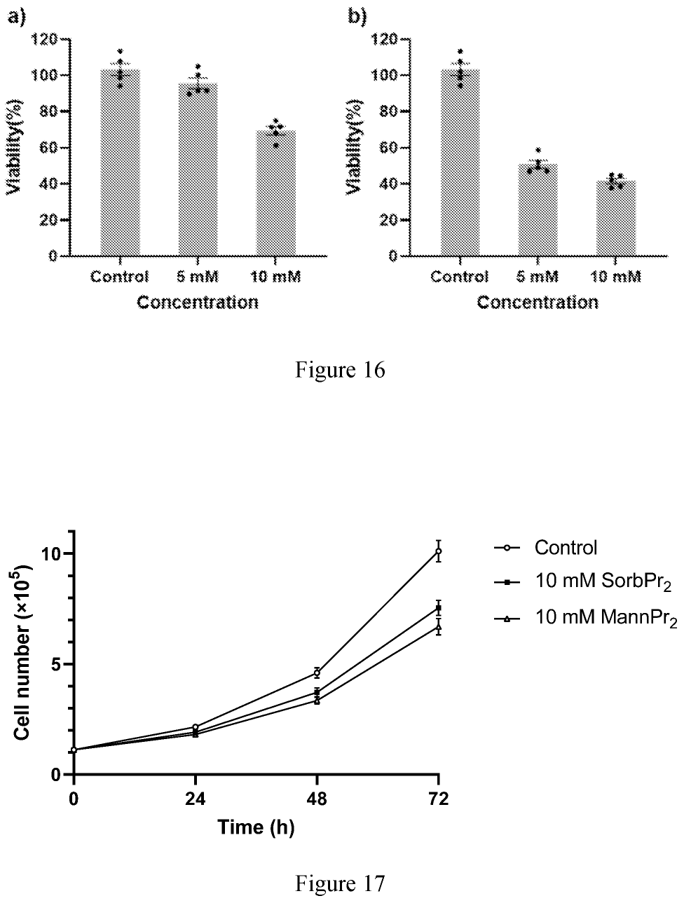

In contrast to the lack of protection from free mannitol and sorbitol, the total recovery values observed for cells incubated with 10 mM of MannPr₂ and SorbPr₂ are much larger than those of cells treated with media only and are comparable to those obtained with cells frozen in media containing 5% DMSO. The protection provided by MannPr₂ is in fact the same as those seen with 5% DMSO. Thus, a sugar alcohol like mannitol or sorbitol, which otherwise does not show enhanced protection for NIH-3T3 cells, becomes as effective as DMSO in protecting cells against freezing damage upon its internalization via its dipropionate ester. The growth of cells incubated with MannPr₂ and SorbPr₂ for up to 72 hrs is slightly suppressed, rather than promoted, relative to that of the control (FIG. 17), indicating that the enhanced post-thaw growth of cells was not due to metabolism enhanced by these esters or the free sugar alcohols inside cells.

Comparing the 0-h, 24-h and 48-h post-thaw total cell recovery values shows that each group of cells, including those stained with calcein-AM (green fluorescence, live cells) and ethidium homodimer-1 (red fluorescence, dead cells). Scale bar 100 μm. incubated only with culture media (control-1), experience a slight drop in their recovery value from 0 h to 24 h, followed by a significant increase from 24 h to 48 h. The cells of control-2, which were first incubated with media followed by freezing in media containing 5% DMSO, showed only a slight increase in their recovery from 0 h to 24 h. Such a reduction in cell recovery was also observed with post-thaw cells in previously reported studies. The stagnant or decreased 24-h post-thaw recovery data as compared to the 0-h values may be due to false positives, i.e., dead cells being counted as lives ones, in the immediate post-thaw numbers. Another reason could be that the treated and frozen cells need a period of time to recover from the shock they experienced from the freezing and thawing process before resuming their growth.

In contrast to the slight reduction in the total cell recovery from 0 h to 24 h post-thaw, the cells incubated with the diesters, especially MannPr₂ and SorbPr_{2,} like those frozen with 5% DMSO, underwent rapid growth from 24 h to 48 h post-thaw. The high recovery and resumed growth of cells beyond 24 h post thaw clearly demonstrate that the majority of cells incubated with diesters MannPr₂ and SorbPr₂ survived the freezing and thawing process, and were as heathy as those cryopreserved with 5% DMSO.

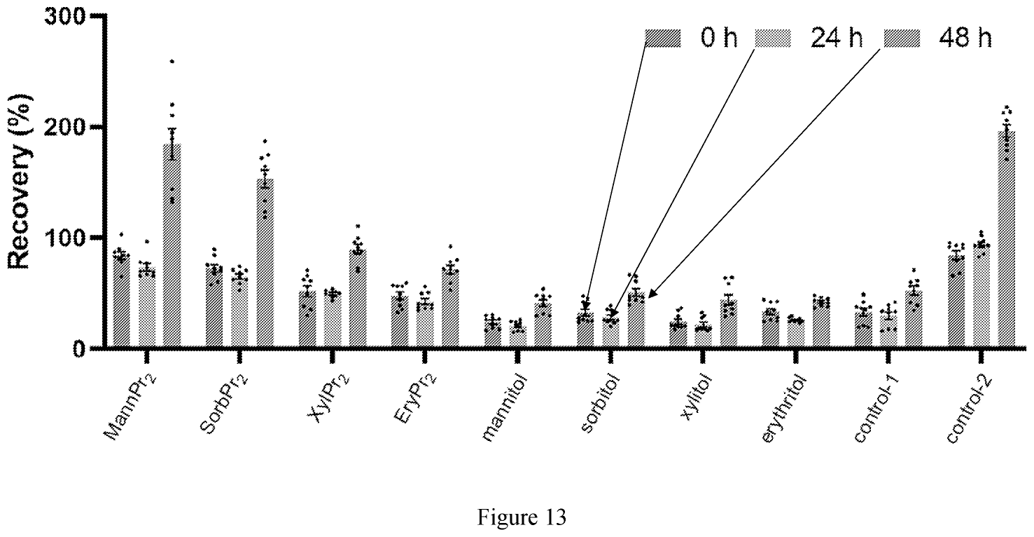

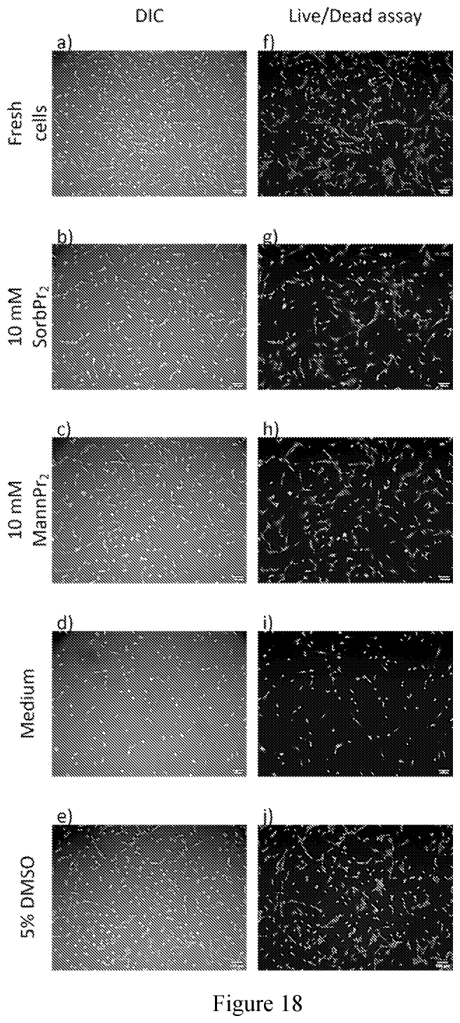

FIG. 14 shows the 24 h post-thaw fluorescence microscopy images of NIH-3T3 cells cryopreserved under the indicated conditions. Micrographs shown in FIG. 14a-c clearly demonstrate that cells incubated with mannitol or sorbitol does not result in higher post-thaw viability than cells treated with media only. In contrast, cells first incubated with diester MannPr₂ (FIG. 14d) or SorbPr₂ (FIG. 14e) before being frozen in media containing 400 mM trehalose show much higher recovery than those treated with mannitol or sorbitol. The cryoprotective effects of MannPr₂ and SorbPr₂ are apparent, with cell viability being much higher than those treated with mannitol and sorbitol, or with medium. Comparing the images of post-thaw cells treated with Sorb-Pr₂ and MannPr₂ and those of resh cells reals no significance difference in morphology (FIG. 18).

In summary, dipropionate esters of sorbitol, mannitol, xylitol, and erythritol, are prepared. Being water-soluble and non-toxic, the modified sugar alcohols are examined as CPAs for preserving NIH-3T3 cells. Initial studies indicate that the diesters, with enhanced hydrophobicity, were able to penetrate cell membranes and get hydrolyzed to release the membrane-impermeable sugar alcohols inside cells. NIH-3T3 cells incubated with all four diesters followed by freezing at −80° C. exhibited enhanced post-thaw viability measured by their total recovery rates which nevertheless differ significantly. The diesters of mannitol and sorbitol, being much more effective than those of xylitol and erythritol, led to post-thaw recovery rates comparable to those preserved with 5% DMSO. Diester of mannitol results in the highest recovery which is the same as that shown by DMSO. The different outcomes observed with the diesters demonstrate that these compounds, and more likely, the free sugar alcohols released inside cells, exercise their cryoprotective capabilities due to their unique molecular structures that results in different interactions with ice nuclei and biomacromolecules. Further study on the molecular and supramolecular factors behind the effects of internalized sugar alcohols on the cryoprotection of cells will provide much needed insights into the corresponding mechanisms, which remain unexplored. The same approach can be extended to the conversion of other readily available sugars, sugar alcohols, and non-toxic compounds into membrane-penetrating derivatives, which could lead to effective intracellular CPAs for DMSO-free cryopreservation.

TABLE 4
Post-thaw cell recovery of NIH-3T3 cells.ª

| | Post-thaw recovery (%) | | |
|---|---|---|---|
| | 0-hr | 24-hr | 48-hr |
| SorbPr₂ (1) | 72.4 ± 3.4 | 65.2 ± 2.2 | 153.2 ± 8.0 |
| (10 mM) | | | |
| MannPr₂ (2) | 84.1 ± 3.3 | 73.6 ± 3.2 | 184.1 ± 14.2 |
| (10 mM) | | | |
| XylPr₂ (3) | 52.1 ± 4.8 | 49.7 ± 1.1 | 90.0 ± 4.3 |
| (10 mM) | | | |
| EryPr₂ (4) | 47.9 ± 3.4 | 42.6 ± 2.6 | 71.6 ± 3.9 |
| (10 mM) | | | |
| Mannitol | 24.1 ± 1.8 | 20.8 ± 1.5 | 41.5 ± 3.0 |
| (10 mM) | | | |
| Sorbitol | 32.8 ± 3.2 | 28.4 ± 2.1 | 50.8 ± 3.5 |
| (10 mM) | | | |
| Xylitol | 24.6 ± 2.4 | 22.1 ± 2.1 | 44.3 ± 4.7 |
| (10 mM) | | | |
| Erythritol | 33.5 ± 2.7 | 26.0 ± 0.6 | 42.3 ± 1.2 |
| (10 mM) | | | |
| Control-1 | 33.2 ± 3.6 | 29.9 ± 3.3 | 52.7 ± 4.0 |
| Control-2 | 84.5 ± 3.8 | 93.9 ± 2.4 | 196.4 ± 5.7 |

^{a}Recovery calculated by trypan blue exclusion. Each value obtained with 3 biological repeats and 3 technical replicates. Errors are ±SEM.

### Experimental Section

Materials and general information. Dulbecco's high glucose modified Eagle's medium (DMEM) with HEPES (25 mM), Penicillin-Streptomycin-Glutamine (100×), PBS (phosphate buffered saline) and trypsin-EDTA (0.25%) were obtained from Gibco. Calcein AM and ethidium Homodimer-1 (EthD-1) for live/dead fluorescence images were obtained from Thermo Fisher. All other chemicals were purchased from commercial sources and used as received. Silica gel for analytical thin layer chromatography (TLC) and column chromatography (mesh 230˜400) were purchased from Sorbent Technologies Inc. 1H NMR spectra were recorded at 300 MHz on Mercury-300 and 400 MHz on INOVA-400. 13C NMR spectra were recorded at 75 MHz on Mercury-300 spectrometers, and 101 MHz on INOVA-400 at ambient temperature using CDCl₃ or DMSO-d₆ as solvents (Cambridge Isotope Laboratories, Inc.). Chemical shifts are reported in parts per million (ppm) downfield from TMS (tetramethylsilane) or the deuterated solvents. Coupling constant in 1H-NMR were expressed in Hertz (Hz). Regular mass spectra (MS-ESI) were recorded on a Thermo Finnegan LCQ Advantage MS spectrometer. High-resolution electrospray ionization mass spectra (HRMS-ESI) and Matrix-assisted laser desorption/ionization (HRMS-MALDI) were recorded on a Bruker SolariX 12 T Fourier Transform Mass Spectrometer.

Cell culture. NIH/3T3 cells (ATCC CCL-92) were cultured in growth medium consisting of Dulbecco's Modified Eagle's Medium (DMEM, high glucose, with 25 mM HEPES) supplemented with 10% fetal bovine serum (FBS), 100 units/mL, 100 μg/mL, 292 ng/mL L-glutamine in an incubator with 5% CO₂ and humidified environment at 37° C. Cells were detached with 0.25% trypsin-EDTA, resuspended in growth medium, and counted prior to passaging.

Cytotoxicity. NIH/3T3 cells were seeded into 96-well plates (Fisher brand) at a density of 8,000 cells in 150 μL growth medium per well. The cells were incubated for 24 hours at 37° C. with 5% CO₂. After 24 hours all medium was aspired one row at a time and treated with various concentrations of each compound (10 mM, 5 mM, 1 mM, 0.5 mM and 0.1 mM) or carboxylic acid/NaOH (10 mM/5 mM, 5 mM/2.5 mM). The control cells were treated with fresh medium only. The cells were then incubated again for 48 hours at 37° C. with 5% CO₂. After incubation, all medium was aspired and replaced with fresh medium and 10% (v/v) CCK-8 reagent and returned to incubate at 37° C. with 5% CO₂ for 2 hours. Cell viability was then calculated from the OD value read by a plate reader (Biotek Synergy H1) at wavelength of 450 nm.

$$\% Cell Viability = \frac{O D treated cells - O D background}{O D control - O D background} ⨯ 100$$

The results of cell viability are calculated with average OD over 5 wells where no data is discarded and error is calculated with the standard deviation among those 5 wells.

Preparation of sample solutions for incubation. Sample solutions with different compounds (diesters and sugar alcohols) were prepared by directly dissolving the compounds into culture medium (DMEM with 10% FBS) and sterile-filtered through a PES 0.2 μm syringe filter.

Incubation (for cell size measurement). NIH/3T3 cells were seeded into 6-well plates at a density of 5×10⁴ cells/mL (2 mL/well), allowed to adhere overnight and then incubated with different sample solutions (2 mL) for 48 hours at 37° C. under 5% CO₂. Control cells were incubated with culture medium.

Cell size measurement. At the end of the incubation, the cells were rinsed with PBS (1 mL) and detached by 0.25% trypsin-EDTA (1 mL). The cells were pelleted (200×g, 5 min) and resuspended in PBS (1 mL). The average cell size of the cell suspension was then obtained with a DeNovix Celldrop cell counter. Bright field/counted image and histogram for one count were attached as an example (FIG. 19). Average cell diameter and statistical analysis were obtained from the histograms.

Cell growth curve. NIH/3T3 cells were seeded into 6-well plates at a density of 5×10⁴ cells/mL (2 mL/well), allowed to adhere overnight and then incubated with different sample solutions (2 mL) up to 72 hours at 37° C. under 5% CO₂. Control cells were incubated with culture medium. After certain time incubation, cells were rinsed with PBS (1 mL), detached by 0.25% trypsin-EDTA (1 mL), pelleted (200×g, 5 min) and resuspended in PBS (1 mL). The cell number were obtained by a DeNovix Celldrop cell counter. The 0 hour was set as the time point just after overnight adhering.

Incubation (for cryopreservation). NIH/3T3 cells were seeded into cell culture treated plates (100 mm) at a density of 2×10⁴ cells/mL (10 mL/plate), allowed to adhere overnight and then incubated with different sample solutions (10 mL) for 48 hours at 37° C. under 5% CO₂. Control cells were incubated with growth medium.

Cryopreservation. At the end of the incubation, the cells were rinsed with PBS (2 mL) and detached using 0.25% trypsin-EDTA (3 mL). The cells were pelleted (200×g, 5 min) and resuspended in growth medium with 400 mM trehalose except for the control-2 group. For control-2 group, cells were resuspended in growth medium with 5% DMSO (v/v). An aliquot of cells of each group was then removed for counting by a DeNovix Celldrop cell counter to obtain prefreeze number of viable cells. The remaining cell suspension was transferred into a 2 mL cryogenic vial. The vials were directly transferred to −80° C. freezer without controlling cooling rate and stored at −80° C. freezer for 3 days. To thaw, cryogenic vials were removed from −80° C. freezer and suspended in a 37° C. water bath until ice melt. To the contents of each vial 1 mL growth medium was added and centrifuged (200×g , 5 min). The supernatant was discarded and cell pellet was resuspended in 1 mL growth cell medium. An aliquot of cell suspension of each group was then removed for counting with a DeNovix Celldrop cell counter to obtain the number of viable cells at 0-hr post-thaw. The remaining cell suspension was split equally and resuspended into two cell culture treated plates (100 mm) with 10 mL growth medium in each plate. The plates were then maintained for either 24 hours or 48 hours at 37° C. under 5% CO₂. After being cultured for either 24 hours or 48 hours, the cells were rinsed with PBS (2 mL) and detached using 0.25% trypsin-EDTA (3 mL). The cells were pelleted (200×g, 5 min) and resuspended in growth medium. The cell suspension was then analyzed by DeNovix Celldrop cell counter to obtain number of viable cells at either 24-hr or 48-hr post-thaw.

Trypan Blue Exclusion Assay. For all time points, a sample of cells was mixed 1:1 in 0.4% trypan blue and counted using a DeNovix Celldrop. Cell recovery was calculated as the ratio of viable cells to the number of cells initially frozen.

$$Recovery ( \% ) = \frac{Viable {Cells}_{post - thaw}}{Viable {Cells}_{prefreeze}} ⨯ 100$$

DIC and Fluorescence images capturing. NIH/3T3 cells were treated as the same as for cryopreservation experiment and were thawed in a 37° C. water bath until ice melt. Cells in each vial were then pelleted (200×g, 5 min), re-suspended in 1 mL fresh growth medium and plated into 96 well plate and grown for 24 hours. Each well was washed with sterile PBS (100 μL). Sterile PBS (200 μL) containing 2 μM calcein-AM and 4 μM ethidium homodimer-1 (EthD-1) was then added to each well. The 96-well plate was then incubated in a CO₂ incubator at 37° C. for 30 min. Fresh cells without freezing were plated at similar density in 96-well plate as comparison. DIC and fluorescence microscopy images were captured at 509 and 580 nm on Axioplan 2 fluorescent microscope (Zeiss [Carl Zeiss], Thornwood, NY) with an Axiocam MRm camera (Zeiss). Axiovision 4 software (Zeiss) was used for image acquisition. Digital images were processed with ImageJ (imagej.nih.gov/ij/).

### Synthesis and Characterization

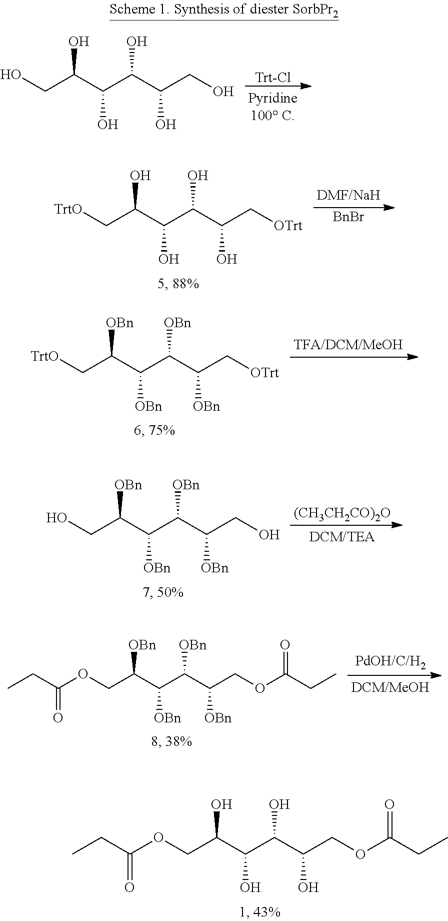

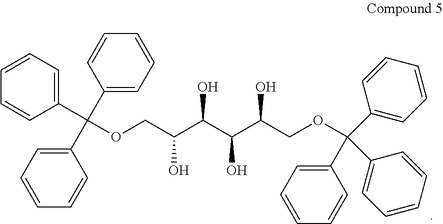 [SMILES: O[C@@H]([C@H](O)[C@@H](O)COC(c1ccccc1)(c1ccccc1)c1ccccc1)[C@H](O)COC(c1ccccc1)(c1ccccc1)c1ccccc1]

To a 500 mL round bottom, sorbitol (5 g, 0.027 mol) was added and dissolved in 60 mL pyridine. Triphenylmethyl chloride (15.8 g, 0.058 mol) was added to the flask, then the mixture was heated to 100° C. overnight. The solvent was then removed under reduced pressure before 100 mL DCM was added and then washed with 50 mL dilute HCl three times. The crude product was then purified via flash chromatography (hexane:ethyl acetate=2:1) to give a white solid product (16.4 g, 88% yield). ¹H NMR (400 MHz, DMSO-d₆) δ 7.51-7.10 (m, 17H), 4.89 (dd, J=41.3, 5.0 Hz, 1H), 4.14 (t, J=6.7 Hz, 1H), 3.83-3.70 (m, 2H), 3.32 (dd, J=16.1, 9.5 Hz, 1H), 3.11 (dd, J=9.3, 2.7 Hz, 1H), 3.07-2.91 (m, 2H). ¹³C NMR (101 MHz, DMSO-d₆) δ 150.0, 144.6, 144.5, 136.6, 128.9, 128.2, 128.1, 128.0, 127.3, 127.2, 124.3, 86.1, 72.7, 72.4, 70.7, 69.5, 66.6, 65.5. MS (ESI-TOF) m/z: [M+Na]⁺ Calcd for C₄₄H₄₂O₆Na 689.8, found 689.5.

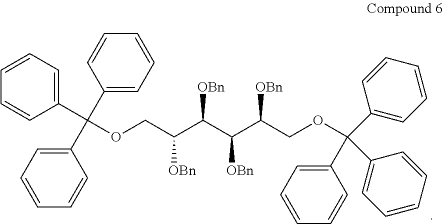 [SMILES: c1ccc(CO[C@@H]([C@H](OCc2ccccc2)[C@@H](COC(c2ccccc2)(c2ccccc2)c2ccccc2)OCc2ccccc2)[C@H](COC(c2ccccc2)(c2ccccc2)c2ccccc2)OCc2ccccc2)cc1]

To a 500 mL round bottom compound 5 was added (7.2 g, 0.0108 mol) was dissolved in 100 mL DMF. Benzyl bromide (6.4 mL, 0.0649 mol) was added before the mixture was cooled to 0° C. Sodium hydride (2.5 g, 0.0649 mol) was then added portion-wise and the reaction was spun overnight. Then water was added slowly and then extracted with 100 mL ethyl acetate 3 times. The organic layer was first dried under sodium sulfate before removing the solvent under reduced pressure before being purified via flash chromatography (hexane:ethyl acetate=10:1) to yield a white solid (8.3 g, 75% yield). ¹H NMR (300 MHz, DMSO-d₆) δ 7.67-6.91 (m, 46H), 4.70 (d, J=11.8 Hz, 1H), 4.51 (dt, J=30.3, 10.4 Hz, 2H), 4.08-3.77 (m, 3H), 3.36 (d, J=8.4 Hz, 1H). ¹³C NMR (75 MHz, DMSO-d₆) δ 148.3, 144.2, 144.2, 139.0, 138.8, 138.3, 128.8, 128.7, 128.5, 128.3, 128.3, 128.2, 128.1, 128.0, 127.8, 127.6, 127.5, 127.4, 127.1, 86.5, 86.3, 81.0, 79.8, 79.3, 79.0, 78.1, 74.1, 73.2, 72.7, 71.8. MS (ESI-TOF) m/z: [M+Na]⁺ Calcd for C₇₂H₆₆O₆Na 1050.3, found 1049.8.

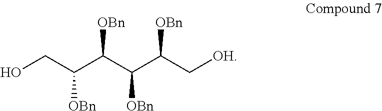 [SMILES: OC[C@H](OCc1ccccc1)[C@@H](OCc1ccccc1)[C@H](OCc1ccccc1)[C@@H](CO)OCc1ccccc1]

Compound 6 (3 g, 0.0029 mol) was dissolved in 30 mL DCM and 60 mL methanol. Trifluoroacetic acid (13.5 mL) was added and allowed to spin overnight. All solvent was removed under reduced pressure before purifying via flash chromatography (hexane:ethyl acetate=1:1) to yield a clear oil (0.450 g, 50%). ¹H NMR (300 MHz, DMSO-d₆) δ 7.26 (dt, J=13.7, 6.4 Hz, 9H), 4.71 (d, J=11.6 Hz, 1H), 4.65-4.35 (m, 3H), 3.96 (s, 1H), 3.83 (d, J=10.2 Hz, 1H), 3.67 (q, J=12.3, 9.9 Hz, 2H). ¹³C NMR (75 MHz, DMSO-d₆) δ 139.4, 128.6, 128.1, 127.9, 127.9, 127.7. MS (ESI-TOF) m/z: [M+H⁺] Calcd for C₃₄H₃₉O₆ 543.7, found 543.2.

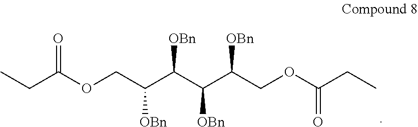 [SMILES: CCC(=O)OC[C@H](OCc1ccccc1)[C@@H](OCc1ccccc1)[C@H](OCc1ccccc1)[C@@H](COC(=O)CC)OCc1ccccc1]

Compound 7 (0.150 g, 0.276 mmol) was dissolved in chloroform, then triethylamine (0.770 mL, 5.52 mmol) was added. Propionic anhydride (0.353 mL, 2.76 mmol) was added slowly then the reaction was spun overnight. The organic layer was then washed with water, followed by saturated potassium bicarbonate, and dilute HCl. The organic layer was then dried over sodium sulfate before removing the solvent under reduced pressure. The crude product was then purified via flash chromatography to give a clear oil (0.070 g, 38%). ¹H NMR (400 MHz, DMSO-d₆) δ 7.26 (q, J=7.2 Hz, 20H), 4.75-4.25 (m, 11H), 4.15 (m, 2H), 3.99-3.74 (m, 4H), 2.24 (m, 4H), 0.97 (m, 6H). ¹³C NMR (101 MHz, DMSO-d₆) δ 173.9, 173.9, 138.8, 138.7, 128.7, 128.6, 128.3, 128.1, 128.0, 127.9, 104.5, 78.7, 78.4, 78.1, 77.4, 74.2, 73.8, 72.5, 71.6, 64.0, 63.3, 27.3, 27.2, 9.4. MS (ESI-TOF) m/z: [M+H^{+]} Calcd for C₄₀H₄₇O₈ 655.8, found 655.6.

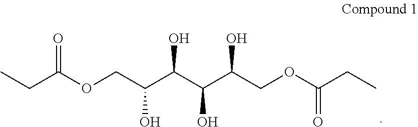 [SMILES: CCC(=O)OC[C@@H](O)[C@@H](O)[C@H](O)[C@@H](O)COC(=O)CC]

Compound 8 (1 g, 0.0015 mol) was dissolved in DCM (20 mL) and methanol (10 mL), to which Pd(OH)₂ (0.1 g, 50% water). The mixture was reacted under hydrogen (50 bar) for at least 6 hours until the starting materials disappeared and only one major spot showing on TLC plate. After removing the catalysis by filtration, the filtrate was concentrated under reduced pressure to remove the solvents. The crude product was purified via flash column chromatography (hexane:ethyl acetate 1:1) to give compound 1 as a white solid (0.193 g, 43%). ¹H NMR (400 MHz, DMSO-d₆) δ 4.93 (d, J=4.9 Hz, 1H), 4.86 (d, J=5.9 Hz, 1H), 4.47 (dd, J=21.2, 6.7 Hz, 2H), 4.23 (dd, J=11.2, 2.6 Hz, 1H), 4.12-3.92 (m, 3H), 3.77 (m, 1H), 3.67 (m, 2H), 3.43 (m, 1H), 2.31 (m, 4H), 1.03 (t, J=7.5 Hz, 6H). ¹³C NMR (101 MHz, DMSO-d₆) δ 174.3, 174.2, 71.5, 71.0, 69.5, 68.9, 66.8, 66.1, 27.3, 9.4. HRMS (ESI-TOF) m/z: [M+Na⁺] Calcd for C₁₂H₂₂NaO₈⁺ 317.1207, found 317.1214.

Compounds 1a, 2, 2a, 3, and 4 were synthesized based on similar procedures used for preparing compound 1.

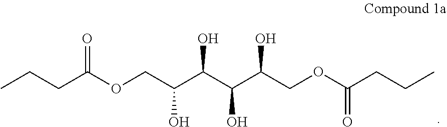 [SMILES: CCCC(=O)OC[C@@H](O)[C@@H](O)[C@H](O)[C@@H](O)COC(=O)CCC]

¹H NMR (400 MHz, DMSO-d₆) δ 4.92 (d, J=5.0 Hz, 1H), 4.85 (d, J=6.0 Hz, 1H), 4.47 (dd, J=19.2, 6.8 Hz, 2H), 4.23 (dd, J=11.3, 2.6 Hz, 1H), 4.15-3.90 (m, 3H), 3.76 (dd, J=7.6, 4.2 Hz, 1H), 3.67 (q, J=5.3, 3.9 Hz, 2H), 3.47-3.36 (m, 1H), 2.27 (m, 4H), 1.55 (m, 4H), 0.88 (td, J=7.4, 1.4 Hz, 6H). ¹³C NMR (101 MHz, DMSO-d₆) δ 173.4, 173.3, 71.5, 71.1, 69.5, 68.9, 66.7, 66.0, 35.9, 35.8, 18.4, 13.9. HRMS (ESI-TOF) m/z: [M+Na⁺] Calcd for C₁₄H₂₆NaO₈⁺ 345.1520, found 345.1533.

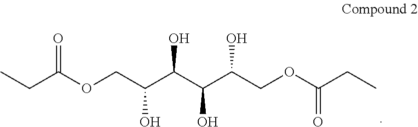 [SMILES: CCC(=O)OC[C@@H](O)[C@@H](O)[C@H](O)[C@H](O)COC(=O)CC]

¹H NMR (400 MHz, DMSO-d₆) δ 4.54 (s, 3H), 4.28 (dd, J=11.2, 2.3 Hz, 2H), 3.97 (dd, J=11.2, 6.5 Hz, 2H), 3.67 (m, 2H), 3.57 (d, J=9.1 Hz, 2H), 2.31 (q, J=7.5 Hz, 4H), 1.03 (t, J=7.5 Hz, 6H). ¹³C NMR (101 MHz, DMSO-d₆) δ 174.3, 69.5, 68.6, 67.3, 27.3, 9.5. HRMS (ESI-TOF) m/z: [M+Na⁺] Calcd for C₁₂H₂₂NaO₈⁺ 317.1207, found 317.1216.

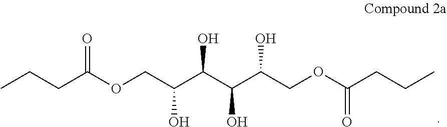 [SMILES: CCCC(=O)OC[C@@H](O)[C@@H](O)[C@H](O)[C@H](O)COC(=O)CCC]

¹H NMR (400 MHz, DMSO-d₆) δ 4.80 (d, J=6.1 Hz, 2H), 4.36-4.25 (m, 4H), 3.96 (dd, J=11.2, 6.6 Hz, 2H), 3.66 (m, 2H), 3.56 (t, J=8.5 Hz, 2H), 3.34 (s, 1H), 2.28 (t, J=7.3 Hz, 4H), 1.55 (m, 4H), 0.88 (t, J=7.4 Hz, 6H). ¹³C NMR (101 MHz, DMSO-d₆) δ 173.5, 69.5, 68.6, 67.3, 35.9, 18.4, 13.9. HRMS (ESI-TOF) m/z: [M+Na⁺] Calcd for C₁₄H₂₆NaO₈⁺ 345.1520, found 345.1538.

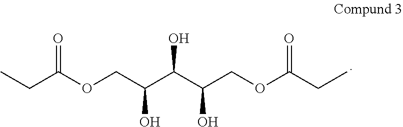 [SMILES: CCC(=O)OC[C@@H](O)[C@H](O)[C@@H](O)COC(=O)CC]

¹H NMR (400 MHz, DMSO-d₆) δ 4.83 (d, J=5.8 Hz, 2H), 4.67 (d, J=6.4 Hz, 1H), 4.09-3.94 (m, 4H), 3.72 (m, 2H), 3.42-3.30 (m, 3H), 2.27 (q, J=7.5 Hz, 4H), 0.99 (t, J=7.6 Hz, 6H). ¹³C NMR (101 MHz, DMSO-d₆) δ 174.1, 71.2, 69.3, 66.0, 27.2, 9.4. HRMS (ESI-TOF) m/z: [M+Na⁺] Calcd for C₁₁H₂₀NaO₇⁺ 287.1101, found 287.1110.

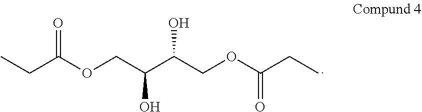 [SMILES: CCC(=O)OC[C@@H](O)[C@@H](O)COC(=O)CC]

¹H NMR (400 MHz, DMSO-d₆) δ 5.08 (d, J=5.1 Hz, 2H), 4.25-4.15 (m, 2H), 3.96 (m, 2H), 3.60-3.52 (m, 2H), 2.32 (q, J=7.5 Hz, 4H), 1.03 (t, J=7.5 Hz, 6H). ¹³C NMR (101 MHz, DMSO-d₆) δ 174.2, 69.6, 66.3, 27.2, 9.4. HRMS (ESI-TOF) m/z: [M+Na⁺] Calcd for C₁₀H₁₈NaO₆⁺ 257.0996, found 257.1011.

### Example 3

This example provides a description of esterified polyols of the present disclosure and methods of using same.

a Modification of sucrose

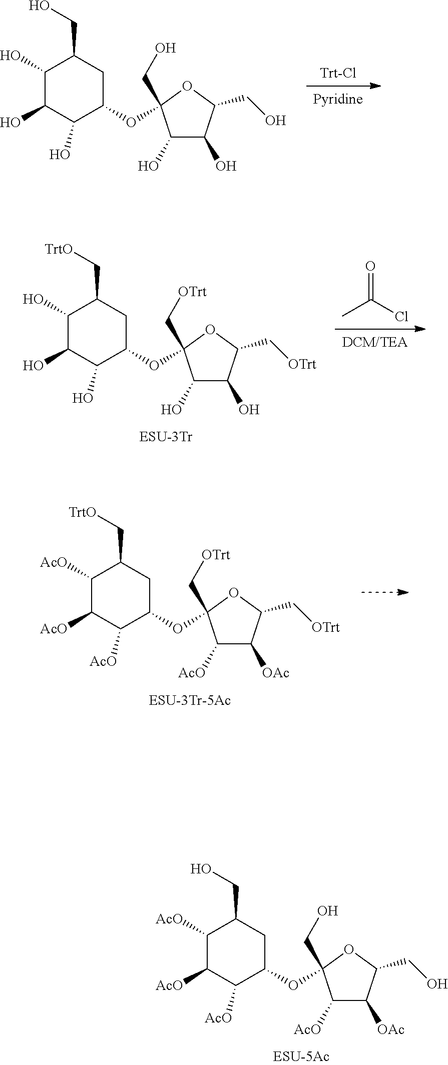

Modification of trehalose

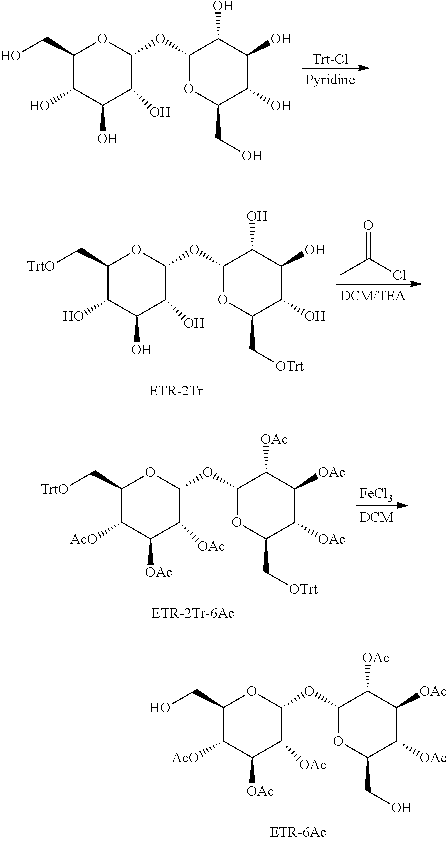

The final product ETR-6Ac has been obtained. The resultant derivatives, after penetrating into cells, are hydrolyzed by intracellular esterases into the original membrane-impermeable forms which are entrapped inside cells.

Conjugates of glycerol and amino acids

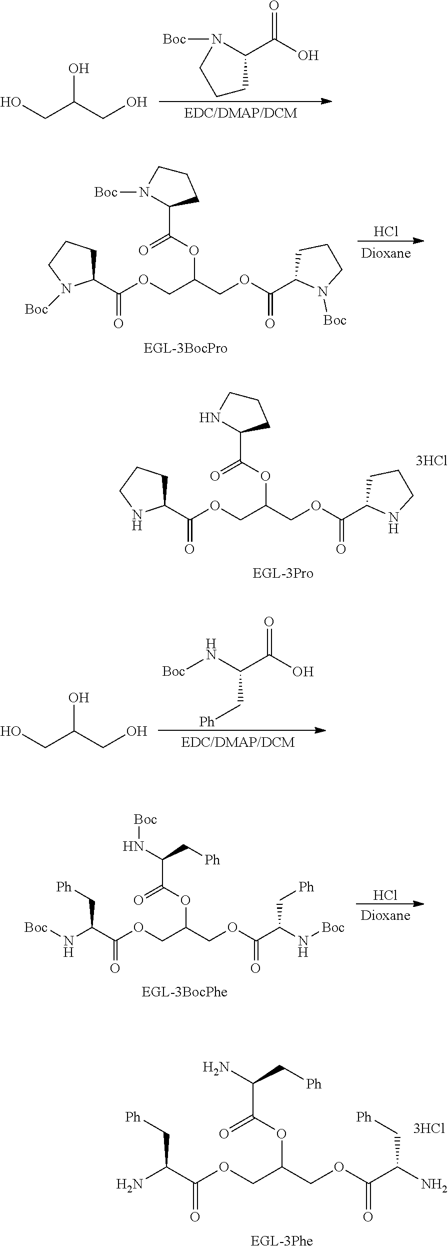

Both EGL-3Pro and EGL-3Phe have been obtained. EGL-3Pro has good water solubility (at least 10 mM at pH 7.4) but EGL-3Phe is just slightly soluble in water (up to 2 mM at pH 7.4).

Conjugates of sorbitol/mannitol and amino acids

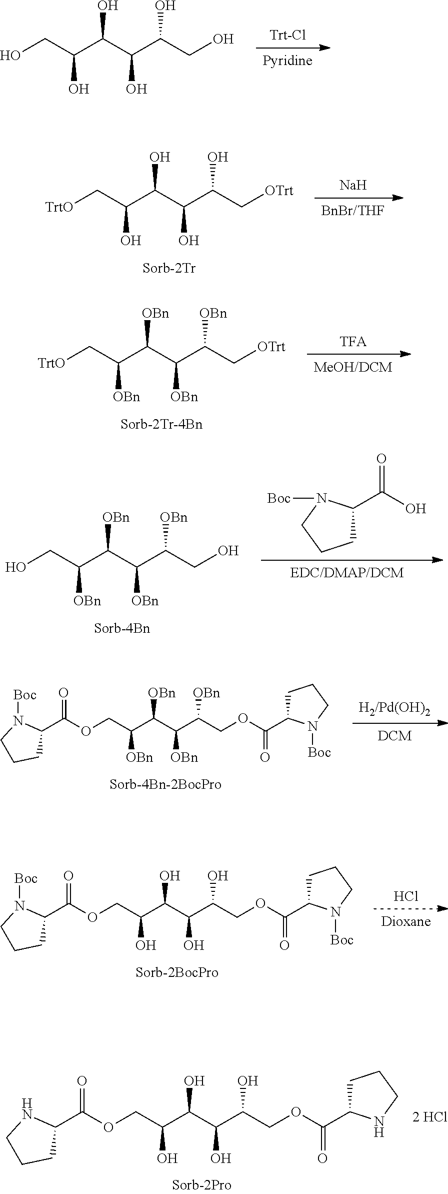

The derivatives of mannitol, Mann-2BocPro, and Mann-2BocPhe, are prepared based on the same procedures shown above:

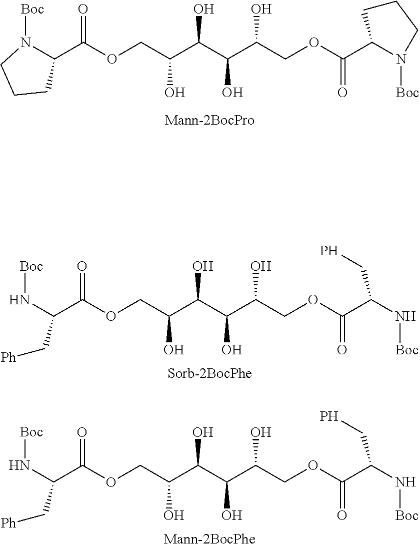

Sorb-2BocPro has been obtained but the final product Sorb-2Pro has not been obtained. Following to the same procedure, Sorb-2BocPhe, and Mann-2BocPhe have all been obtained. The Boc groups of these compounds will be removed with HCl/dioxane to give Sorb-2Phe and Mann-2Phe which will be directly used for cryoprotection without being stored.

Cytotoxicity: Cytotoxicity of EGL-3Pro and EGL-3Phe were tested by CCK-8 kit on 3T3 cells after incubating for 48 hours at different concentrations. EGL-3Pro didn't show any toxicity up to 10 mM. EGL-3Phe showed apparent cytotoxicity above 1 mM, residual cytotoxicity at 0.5 mM and no toxicity at 0.1 mM.

Cryopreservation: The cryopreservation effects of EGL-3Pro and EGL-3Phe were tested on 3T3 cells. The cells were incubated with 5 mM or 10 mM EGL-3Pro or 0.5 mM EGL-3Phe or medium only (two control groups) for 72 hours. After incubation, the cells were detached by treating with 0.25% and pelleted by centrifuging (200×g). The cells were then resuspended in medium with 400 mM trehalose or 5% DMSO (for control 2 group) in cryovials and a small portion of cell suspension were used for counting pre-freeze cell number. The vials were directly put in −80° C. freezer and stored for at least 3 days. The cells were thawed in 37° C. water bath until ice melt, and resuspended in 10 mL fresh medium and pelleted by centrifuging (200×g). The cells were then resuspended in 1 mL fresh medium and counted by cell counter. The recovery rate was obtained by dividing post-thaw lived cell number by pre-freeze lived cell number.

TABLE 5
Post Thaw Recovery

| Entry | 0-hour post thaw recovery (%) |
|---|---|
| 10 mM EGL-3Pro | 65.3 ± 7.3 |
| 5 mM EGL-3Pro | 50.1 ± 6.6 |
| 0.5 mM EGL-3Phe | 66.2 ± 10.1 |
| Control 1 (medium only) | 51.7 ± 9.1 |
| Control 2 (5% DMSO) | 77.9 ± 3.8 |

Although the present disclosure has been described with respect to one or more particular embodiments and/or examples, it will be understood that other embodiments and/or examples of the present disclosure may be made without departing from the scope of the present disclosure.

1. An esterified polyol, wherein the esterified polyol has the following structure: 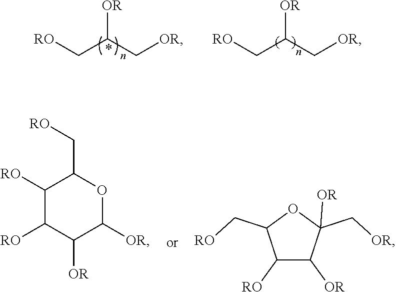
wherein n is 1, 2, 3, or 4 and each R is independently H or 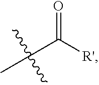
wherein each R′ is independently chosen from aliphatic groups, aryl groups, and amino acid groups and at least one R is not H, wherein the asterisked carbon has R stereochemistry or S stereochemistry, and
when the esterified polyol has the following structure: 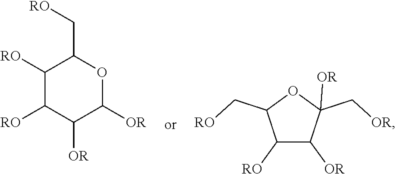
one or more of the carbons have R or S stereochemistry.

2. The esterified polyol according to claim 1, wherein each aliphatic group is chosen from a CH₃ group, a C₂H₅ group, linear and branched C₃H₇ groups, linear and branched C₄H₉ groups, linear and branched C₅H₁₁ groups, and linear and branched C₆H₁₃ groups.

3. The esterified polyol according to claim 1, wherein the aryl group is a phenyl group or 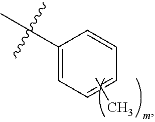
wherein m is 1, 2, 3, 4, or 5.

4. The esterified polyol according to claim 1, wherein the amino acid group is a canonical amino acid group, a non-canonical amino acid group, or an N-functionalized glycine-based derivative.

5. The esterified polyol according to claim 1, wherein the esterified polyol has the following structure:

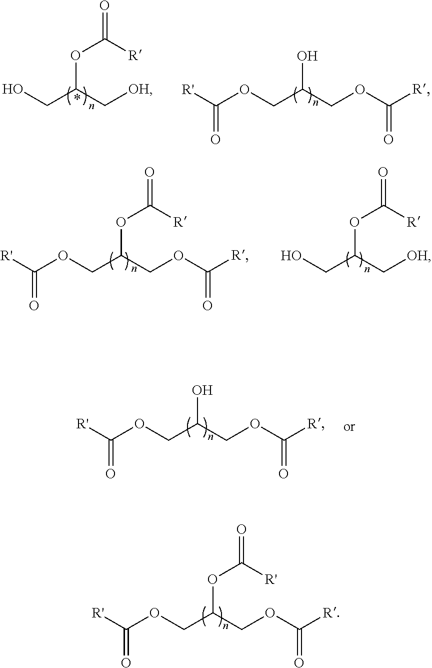

6. The esterified polyol according to claim 5, wherein the esterified polyol is: 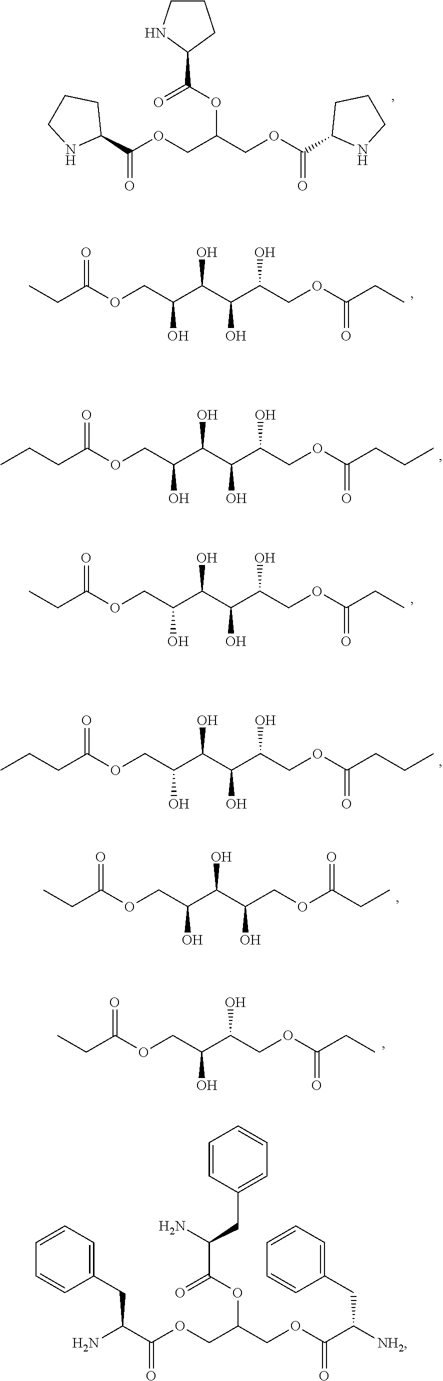 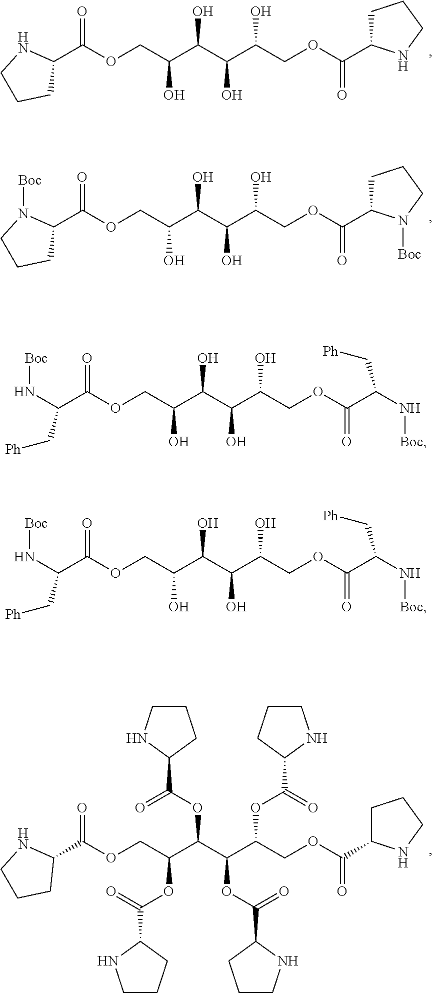
or salt thereof.

7. The esterified polyol according to claim 6, wherein the esterified polyol is:

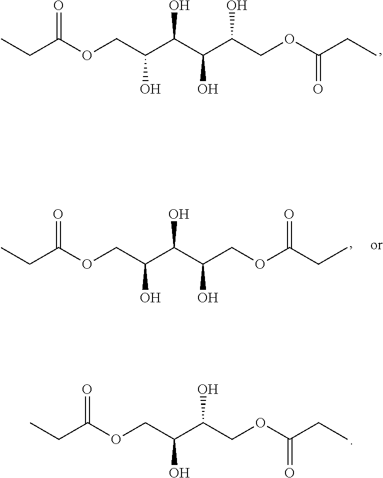

8. The esterified polyol according to claim 1, wherein the esterified polyol is:

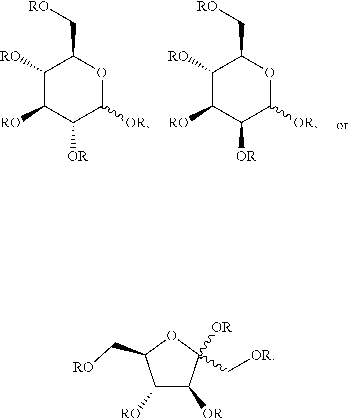

9. An esterified polyol having the following structure: 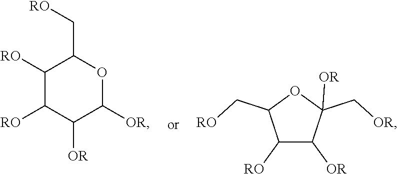
wherein n is 1, 2, 3, or 4 and each R is independently H, a saccharide group, or 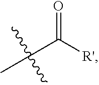
wherein each R′ is independently chosen from aliphatic groups, aryl groups, and amino acid groups and at least one R is not H and one or more of the carbons have R or S stereochemistry.

10. The esterified polyol according to claim 9, wherein the disaccharide is sucrose or trehalose.

11. The esterified polyol according to claim 9, wherein the esterified polyol has the following structure:

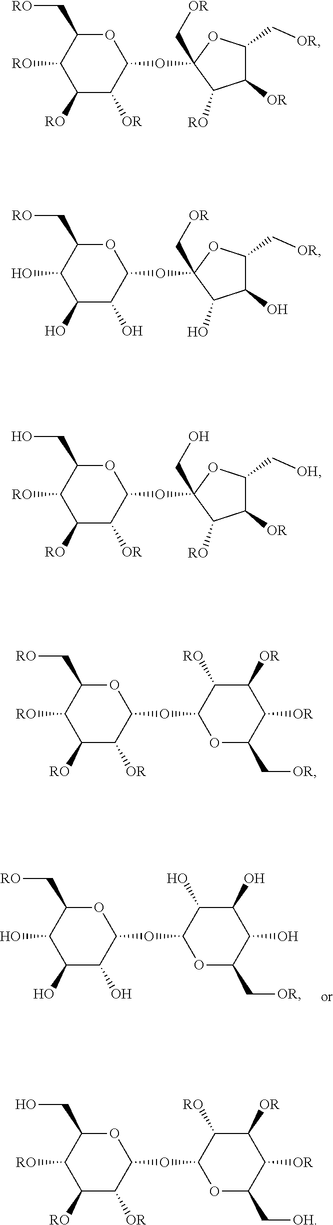

12. The esterified polyol according to claim 9, wherein each aliphatic group is chosen from a CH₃ group, a C₂H₅ group, linear and branched C₃H₇ groups, linear and branched C₄H₉ groups, linear and branched C₅H₁₁ groups, and linear and branched C₆H₁₃ groups.

13. The esterified polyol according to claim 9, wherein the aryl group is a phenyl group or 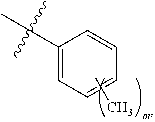
wherein m is 1, 2, 3, 4, or 5.

14. The esterified polyol according to claim 9, wherein the amino acid group is a canonical amino acid group, a non-canonical amino acid group, or an N-functionalized glycine-based derivative.

15. The esterified polyol according to claim 11, wherein the esterified polyol has the following structure:

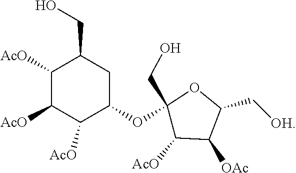 [SMILES: CC(=O)O[C@H]1[C@H](OC(C)=O)[C@@H](CO)C[C@H](O[C@]2(CO)O[C@H](CO)[C@@H](OC(C)=O)[C@@H]2OC(C)=O)[C@@H]1OC(C)=O]

16. A composition comprising an esterified polyol according to claim 1 or claim 9.

17. The composition according to claim 16, further comprising a buffered aqueous solution.

18. A method for cryopreserving a cell population comprising:
contacting the cell population in a suspension with an esterified polyol according to claim 1 or claim 9,
freezing the suspension, wherein the cell population is cryopreserved

19. The method according to claim 18, wherein the concentration of the esterified polyol in the suspension is 0.1 to 100 mM.

20. The method according to claim 19, wherein the concentration of the esterified polyol is 5 mM or 10 mM.

21. A method for preparing a cell population for cryopreservation comprising contacting a population in a suspension with an esterified polyol according to claim 1 or claim 9, wherein the cell population is prepared for cryopreservation.

22. The method according to claim 20, wherein the concentration of the esterified polyol is in the suspension is 0.1 to 100 mM.

23. The method according to claim 22, wherein the concentration of the esterified polyol is 5 mM or 10 mM.

24. A method for moisturizing skin comprising contacting a desired area of an individual with a composition according to claim 16.

25. The method according to claim 24, wherein the contacting comprises topically applying the composition.

26. The method according to claim 24, wherein the composition is an emulsion.

27. The method according to claim 24, wherein the composition is an oil-in-water emulsion.

28. The method according to claim 24, wherein the composition is a cream or lotion.

29. The method according to claim 24, wherein the composition is a solution.
